SEQUENCE LISTING (38 sequences)

SEQ ID NO: 1 - protein, 340 aa, Sorghum bicolor
MEMPPQQAAAAAANANANANGAAHGQDEGGSSSPPLSPASAALANARWNPTKEQVAVLEGLYEHGLRNPSAEQIQQITGRLREHGAIEGKNVFYWFQNHKARQRHRQKQDSFAYFTTLLRRPPPLPVLPMQPAPPYLHGRVPSPSPSPAPAPTAIAMPPPPPPPSPAACNNNNNNNNNGGARVMYRNAFYMPAPQAPPANAAYYYPPPQQQVTVMYQYPRMELGQDKMMMTTAAAQQYPAVMFQQAGAAATNGAGPAERVGPRRETLELFPLKPTFVLRRDKGRAGGGSASALTSASTATASASASEESEGLESGNCNGEEQEAPALPFYDFFGLQSGGR

SEQ ID NO: 2 - protein, 328 aa, Miscanthus lutarioriparius
MAANVGTGRSAGGGGGAGGGTATGSVATAVCRPSGSRWTPTPEQIRILKELYYGCGIRSPNSEQIQRITAMLRQHGKIEGKNVFYWFQNHKARERQKRRLTNLDVNVPAAAAAGAADASTHLGVLSLSSPSGAAPPSPTLGFYAGNGGAGSTVLLDTSSEWGGSAAMATETCFLQDYMGVMGTGSAGAAASPWASFSSSDTMAAAAARAPTVTRAPQTLPLFPTGGDDSQPRRPRHGVPVPAGETIRGGSLPFWGAAPTTASPTASATSVGIQQQHQLLQLQEQYSFYSNTTQRPGTGNQDASAAAAAAASLELSLSSWCSPYPAGTM

SEQ ID NO: 3 - protein, 331 aa, Setaria italica
MEAAPQHGGAGVIDLEGSGSGGAPLSPPLSPASAAAAALANARWNPTKEQLAALEGLYEHGLRTPSAEQIKQITARLREHGHIEGKNVFYWFQNHKARQRQKQKQDSFAYFTRLLRRPPPLPMLVRPPGPLPYPHGRLPVPAPPAAAAAACSSSNGGTHVMYRSPFYMPAAPQAPAANALYYHPQHQHQVPASVMYPRMEVAQDKMIPAAAQHNHHHHRHHPAAGAMYHAAAAGNNASTTPMHVLHFPPAAAVDAGPSRETLQLFPLQPTFLLPADKGRAAGGGASMAPAPSTASASFSGESESLEESPDSYSEALAVPFYDFFGLQSGGR

SEQ ID NO: 4 - protein, 340 aa, Dichanthelium oligosanthes
MEAPQQGSSVVIDLESSGGGAPVSPPLSPASAAAAALANARWNPTKEQLAVLERLYEHGMRTPSAEQIKQITARLREHGHIEGKNVFYWFQNHKARQRQKQKQESFAYFTRLLRRPPPLPMLFRPPGQPYPHGAGRLPVPAAPAAAPVMATMAPPPSPVGSSRNNNGGTHVMYNRAPFYMAAPQGPAANAYYHHMQQQQQHQQQTAVMYPRMEIAQDKMIPAVAQQHQHQHHQAGAMYQAAAAPPPNSASSTPLHVVQHFPAPGADAGPSRRETLQLFPLQPSYLLPDKGLAAAAAGGAHLASASASFSGESEGPASPDNNDEAPALPFYDFFGLQSGGR

SEQ ID NO: 5 - protein, 707 aa, Panicum hallii
MATVNNWLGFSLSPQDLPPSGQTDSSPISAAAADEVSGDVCYNIPQDWGMRGSELSALVSEPKLEDFLGGINFSEQHHKANLNVIPSSSSSTCYASSGASTGYHHQLYHHPSSALHFADSVMVASSAGIHDGGAMLSAATANVGGAGAAGANGGSSIGLSMIKNWLRSQPAPPPQPRVAVAEGAQAAQGLSLSMNMAGSTQGGAGMPLLAGERGRASESVSTSAQGGAVAAARKEDSGGSGGAGALVAVSTDTGGSGGASAETVARKTVDTFGQRTSIYRGVTRHRWTGRYEAHLWDNSCRREGQTRKGRQVYLGGYDKEEKAARAYDLAALKYWGPTTTTNFPVSNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVASYDVGRITSHLGDGSALAAYGSHYHAAAAAGWPTIAFQPGGTAGLYHPYAQPLPRGWCKQEQDNAVIAAAHNLQDLNHLHLGAGGAHDFFSAGQAAAMHGLGSIDNSSLEHSTGSNSVVYNGVGDGNGGSAGGGFLMTMSAAAATATAVASHEQVHARAHGDHDEASKHAAQMGYESYLMNAEAAYGSGRMPSWTPASAPPVAAAASSSDNIAGVGHGGAQLFSVWNDT

SEQ ID NO: 6 - protein, 700 aa, Setaria italica
MATVNNWLGFSLSPQDLPPSQTDSTLISATATDEVSGDVCYNIPQDWSMRGSELSALVAEPKLEDFLGGINFSEQHHKANLNVIPSSSNACYASSGSSTGYHQLYHHQSPALQFADSVMVASSAGVHDGGASMLSAAATVNGGAGAAGANGGSIGLSMIKNWLRNQPAPPLPQPRVVAAAEGAQAAQGLSLSMNMAGAQGSGMPLLAGERGRAPESVSTSAQGGAVAPRKEDSGSSGGAGALLAVSTDTGGSGTVAETAAKKTVDTFGQRTSIYRGVTRHRWTGRYEAHLWDNSCRREGQTRKGRQVYLGGYDKEEKAARAYDLAALKYWGPTTTTNFPISNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILESSALPVGGAAKRLKDAEAAASAQQHAGVVSYDVGRIASHLGDGSALAAYGTHYHAAAAAWPTIAFQPSAAAAGLYHPYAQPMRGWCKQEQDHAVIAAAHSLHELNHLHLGAGGGAHNFFSAGQQAAMHGLGSIDNSSLEHSTGSNSVVYNGVGDGNGGAVGGGYMMPMSAATATTTAMVSHEQAHARSHQGEQDEATKQAAQMGYESYLVNAEAAYGGGRMPSWTPASAPAAASSNDNMAGVGHGGAQLFSVWNDT

SEQ ID NO: 7 - DNA, 1023 bp, Sorghum bicolor
  source 1..1023: mol_type = other DNA
atggagatgcccccccagcaggcggccgccgcggcagccaacgcaaatgcgaacgcaaacggcgctgcacacgggcaagacgagggcggctccagcagccctcctctttcccctgcgtctgcagcgcttgccaatgcgcgctggaatccaacgaaagagcaggtggccgtcctcgagggcctctacgaacatggcctgcgcaatccttctgccgagcaaatccagcagatcactggaaggctccgggagcacggcgccattgagggaaagaacgtgttttactggttccaaaatcacaaggcccggcagcggcacaggcaaaagcaggactccttcgcttacttcacaacacttctgcgcaggccgccgccgctgccagttctgcccatgcaacctgccccgccctatttacatggcagggtccccagcccgtcccccagccctgcgcctgctcctactgctatagccatgccaccaccgccgccgcctccatccccggcagcttgcaacaacaacaacaacaataataacaacgggggtgccagggtcatgtaccggaacgctttctatatgccagcgccacaggctccaccggcgaacgcggcctattactatccccccccacaacagcaagtgacagttatgtaccagtacccgcgcatggagctcggccaggacaagatgatgatgactaccgccgccgcccagcagtatcctgcagtgatgttccagcaggccggagcggcggctaccaatggcgctggtcccgcggaaagggtcggtccaaggcgcgaaacgctggaacttttccccctcaagccaacctttgttctccgccgggataaaggcagggctgggggtggatctgcatccgcgctgaccagcgccagcacggctactgccagcgcaagcgcctctgaggaatccgagggactcgagtctgggaattgtaacggtgaggagcaggaagctccggcactgccgttctacgatttttttggcctccaatctggcgggcgctga

SEQ ID NO: 8 - DNA, 987 bp, Miscanthus lutarioriparius
  source 1..987: mol_type = other DNA
atggctgctaatgttggcacgggccgctccgcgggtgggggtggtggggccggcggcggaactgcaacgggaagcgttgcgaccgctgtctgccggccctccgggtccaggtggacccctaccccggaacagatcaggatcctcaaggaactctattacggatgtggcatccgctcccctaactctgagcaaattcaacgcattaccgcgatgctcaggcagcacggcaaaatcgaggggaagaacgtcttttactggtttcagaatcacaaggctcgggagaggcagaagcgccgcctcacaaacctcgacgtgaacgtccctgcagcagcagcagctggtgccgccgatgcttctacgcatcttggtgtgctgtctctgtcctcccccagcggcgcagcgccaccctctccgacactgggattctacgccggcaacggcggcgccggaagcacagtgcttctggatacctcttctgagtgggggggttctgcggccatggctaccgagacttgctttctccaagactatatgggcgtgatgggaactggaagcgccggcgccgccgcgagcccatgggcctccttctccagctccgacacaatggccgctgcggcggcgagggctccaactgtgactcgggcgccacagactctgcccctgttcccgacggggggcgatgactctcagccacgccggccgaggcatggggtccctgtccctgctggcgaaaccataaggggtggcagcctgcccttctggggcgccgccccgacaacggcaagccccaccgccagcgccaccagcgttggcatacagcagcaacaccagctccttcagctccaagagcagtatagcttctacagcaatactacacaacggccgggtaccggcaaccaagacgcctccgccgccgctgcggcggcagcaagccttgaactttctctgtcctcctggtgctctccatacccggcggggacgatgtga

SEQ ID NO: 9 - DNA, 996 bp, Setaria italica
  source 1..996: mol_type = other DNA
atggaggctgccccacaacatggcggagcaggggttattgatctcgaaggcagcggttctgggggggcacccctctcccccccactttctccggctagcgcagcggccgctgccctcgccaatgcacggtggaatccaactaaggaacagctcgctgccctggaaggcctctacgagcacggccttcgcacacctagcgccgagcaaattaaacagataaccgcccggctcagggagcacggacacatcgagggcaagaacgtgttctattggttccagaaccacaaggccaggcagcgccagaagcaaaagcaggacagcttcgcttactttactcgcctgctgcggaggcccccaccgctgccgatgctcgttcggccaccgggcccgctgccctacccccatggtaggctccctgtgcctgcgccccctgccgccgcggctgcagcgtgctcctccagcaacggcggaacccatgtgatgtaccggtccccattttatatgccagcggctccgcaagccccggccgccaatgcgctctactaccatccacagcaccaacatcaggtgccagcatctgtcatgtatcccaggatggaggtggcgcaggataagatgatcccagcagcggcccaacacaaccaccatcaccatcgccatcaccctgcagccggcgcaatgtaccacgcagccgcggccggtaacaacgcgtctacaaccccgatgcacgtcctgcacttcccgcctgcggccgcggtcgacgctggtccgtcccgcgaaacgcttcagcttttccccctgcagccgacgtttctgcttcctgctgacaaaggcagggcggccggtggaggggcttctatggcgcctgctcccagcactgcctccgcgtctttttccggcgaatccgagtccctggaggagagccctgacagctattctgaagctcttgctgttccgttctacgatttcttcggccttcagagcggagggcgctga

SEQ ID NO: 10 - DNA, 1023 bp, Dichanthelium oligosanthes
  source 1..1023: mol_type = other DNA
atggaagcgccccaacagggctcctccgtcgtcatagaccttgagagcagcggcggcggtgctccggtttccccgcctctcagcccggccagcgcggcggctgctgctctggcgaacgcaaggtggaaccccacaaaggaacagctggcagtgcttgaacgcctctacgaacacggcatgcggacgccatctgcagagcagattaaacagatcacagccaggcttcgggagcatgggcacatcgagggcaagaacgtgttctactggtttcagaaccacaaggcgcgccaaaggcagaagcagaagcaagagagctttgcctatttcactcggctcctcaggcgccccccgcctctcccaatgctcttccgccccccaggtcaaccgtacccacacggtgctggtcgccttcccgtccctgccgcgcctgccgccgctccggtgatggccaccatggcccccccgccatctcctgtcggatcctctaggaataacaatgggggcactcatgtgatgtacaatcgcgcacccttttatatggccgcgccgcagggccctgctgcgaacgcgtactatcatcatatgcagcaacaacagcaacatcagcaacagaccgcggtgatgtaccctaggatggaaattgcacaggataagatgatcccagccgttgcccagcagcaccaacaccagcaccaccaggctggcgccatgtaccaagccgcggcagcgccgcccccaaattctgcgtcctccacccccctgcatgtcgttcagcacttcccagcccccggagcggatgctggtccgtccaggcgggaaacgcttcagctgttcccgctccaaccatcttacctgctgccagacaaaggccttgccgctgccgccgcagggggcgcacacctggccagcgcttctgctagcttctccggcgagagcgagggaccggcaagccctgacaacaacgatgaggcccctgcactcccattttatgacttcttcggactgcagtctggagggcggtga

SEQ ID NO: 11 - DNA, 2124 bp, Panicum hallii
  source 1..2124: mol_type = other DNA
atggccactgtcaacaattggctcggattttccctcagcccacaagatctccctccaagcggccaaaccgattccagcccaatttccgccgctgccgctgatgaagtttccggcgatgtctgttacaacatccctcaagattggggaatgaggggcagcgagcttagcgctcttgtttccgagcctaagcttgaggatttcctcggcggcatcaattttagcgagcagcaccacaaggccaaccttaatgtcatccctagctccagctccagcacttgctatgctagctccggagcctccaccggctaccatcaccagctctatcatcacccttcctccgcccttcacttcgctgacagcgttatggtcgcctcctccgccggaattcatgacggaggcgccatgctctccgccgctactgctaacgttggaggcgccggagccgctggagctaatggaggatccagcatcggcctctccatgattaagaattggctccgcagccaaccagccccacctccacagccacgcgtcgccgttgctgagggcgcccaagccgcccaaggcctctccctcagcatgaatatggctggctccactcaaggcggcgccggaatgcctctcctcgctggcgaaaggggacgcgcttccgagtccgtcagcaccagcgcccaaggaggagccgttgccgctgctaggaaggaggatagcggcggcagcggaggcgctggcgcccttgttgccgtttccactgacactggaggatccggaggcgctagcgctgaaaccgttgctcgcaaaactgttgataccttcggacagaggactagcatttatcgcggcgttactcgccaccgctggactggacgctatgaggctcacctctgggataatagctgtcgccgcgaaggacaaacccgcaagggacgccaagtctaccttggcggctatgacaaggaggaaaaggctgctagggcctatgatcttgccgccctcaagtattggggccctaccaccaccactaactttccagtcagcaactatgagaaagagcttgaggagatgaagcacatgacccgccaagagttcgtcgccagccttcgccgcaaatccagcggcttctctcgcggcgcctccatctataggggcgtcacccgccaccatcaacatggccgctggcaagctcgcatcggaagggttgctggcaacaaggatctctatctcggcaccttcagcacccaagaggaggctgctgaggcctacgacatcgccgccattaaattccgcggactcaacgccgtcactaacttcgacatgtcccgctatgacgtcaagagcatccttgacagcagcgcccttcctattggatccgccgctaagcgcctcaaagaagccgaggccgctgctagcgctcaacaccatgctggcgtcgcctcctatgatgttggccgcatcacttcccaccttggcgatggcagcgctcttgccgcttacggcagccattatcatgccgctgccgccgctggctggcctactatcgctttccagcccggcggcaccgccggactttaccatccttacgcccagccacttcctcgcggatggtgcaagcaagagcaagataacgctgtcatcgctgctgctcataacctccaagatcttaaccacctccaccttggagccggcggagcccatgatttttttagcgccggccaagctgccgctatgcatggacttggaagcatcgacaactccagccttgagcattccactggctccaactccgtcgtttacaatggcgttggcgatggcaatggcggatccgccggaggcggcttcctcatgactatgagcgccgctgctgctaccgctaccgctgttgccagccatgagcaagtccatgctagggcccacggcgatcacgacgaagccagcaaacatgctgcccagatgggctacgagtcctacctcatgaacgccgaagctgcctacggcagcggacgcatgcctagctggaccccagccagcgcccctccagttgctgctgccgccagcagcagcgacaatatcgccggagtcggacatggcggcgcccagctcttttccgtctggaacgacacctga

SEQ ID NO: 12 - DNA, 2103 bp, Setaria italica
  source 1..2103: mol_type = other DNA
atggctactgtcaacaattggctcggcttttccctctccccacaagatctcccaccatcccagaccgactccaccctcatcagcgctactgccactgatgaggtcagcggcgacgtctgctacaatatcccacaagattggtccatgcgcggcagcgaacttagcgctcttgtcgctgagcctaagcttgaggatttccttggcggcatcaacttcagcgagcaacatcacaaggctaacctcaacgtcatccctagctccagcaacgcttgttatgcctcctccggatcctccactggctatcaccagctctatcaccatcagagcccagccctccagttcgccgatagcgtcatggtcgcttccagcgctggcgttcatgacggaggcgctagcatgctttccgctgctgccactgttaatggaggagctggagctgctggcgctaatggcggaagcattggcctcagcatgatcaagaattggcttcgcaaccagccagcccctcctctcccacagcctagggtcgtcgctgctgctgagggcgctcaagctgcccaaggactctccctttccatgaacatggccggcgctcaaggcagcggaatgccactccttgctggagagaggggacgcgctccagagtccgtttccacttccgcccaaggcggagctgttgccccacgcaaggaggattccggaagcagcggaggagccggcgctctcctcgctgtttccaccgacaccggcggatccggaactgtcgctgaaaccgccgccaaaaagaccgttgacacctttggccagcgcaccagcatctatcgcggagttacccgccaccgctggactggacgctatgaagcccacctctgggacaatagctgtcgccgcgagggacagactcgcaagggacgccaagtctaccttggcggctatgataaggaggagaaggccgctcgcgcttacgaccttgctgcccttaaatactggggccctaccaccaccaccaacttccctatctccaattacgagaaggagcttgaggagatgaagcacatgactcgccaagagttcgtcgcttcccttaggcgcaagagcagcggcttctctcgcggcgcttccatctaccgcggagtcacccgccaccatcagcatggccgctggcaagctaggattggacgcgtcgccggcaacaaggacctttacctcggaaccttcagcactcaagaggaggccgctgaggcctacgatatcgccgccatcaaatttcgcggcctcaacgctgtcaccaacttcgatatgtcccgctacgacgtcaaatccatccttgaaagcagcgccctcccagtcggcggagccgctaagaggcttaaggatgccgaggctgctgcttccgcccagcagcatgctggcgttgtctcctacgatgtcggaaggattgccagccatcttggcgatggcagcgctctcgctgcttacggcactcattaccatgccgctgctgccgcttggccaaccatcgctttccagcctagcgctgccgctgccggactctatcatccttatgcccagcctatgaggggctggtgcaagcaagagcaagatcatgctgtcatcgccgccgcccatagcctccacgagcttaatcacctccatctcggcgctggcggaggagcccataactttttttccgccggccagcaagctgctatgcacggccttggaagcatcgacaatagcagccttgagcatagcaccggaagcaactccgtcgtctacaatggcgtcggcgatggaaacggaggagccgtcggaggaggatacatgatgcctatgagcgccgccactgctaccactaccgccatggtttcccatgaacaagcccacgctcgctcccaccaaggagagcaagatgaagccaccaagcaagccgcccagatgggctatgagtcctatcttgtcaacgccgaggctgcctatggcggcggaaggatgcctagctggactccagccagcgccccagccgccgctagcagcaacgacaatatggccggagttggccatggcggagcccaactcttcagcgtttggaacgatacttga

SEQ ID NO: 13 - protein, 6 aa, synthetic construct
TLPLFP

SEQ ID NO: 14 - protein, 5 aa, synthetic construct
ASLEL

SEQ ID NO: 15 - protein, 7 aa, synthetic construct
TLELFPL

SEQ ID NO: 16 - protein, 7 aa, synthetic construct
TLQLFPL

SEQ ID NO: 17 - protein, 328 aa, Andropogon chinensis
MEMPPSAAFAGQDEGGSSPPLSPASAALANARWNPTKEQVAVLEGLYEHGLRNPSSEQIQQITGRLREHGAIEGKNVFYWFQNHKARERQREKQDSFAYFTRLLRRPPPLPVLSSMPPAPLPVLSSMPAAPPYHRSRVPSPAPAPAPAAIAMPPPPPPPSPAACNNNNNNGGARVMYRNAFYMPAPQAPPANAAYYYPPPHQQVTVMYQYPRMELGQDKMMTMTPAAAMFQQAAAAPHFQPLANGGVGPAERVGRSRETLELFPLQPTFVLRRDKGRAEAGCSGSASQTSASTSISEESEGLESGNCNGGEALALPFYDFLGLQSGGR

SEQ ID NO: 18 - protein, 335 aa, Cymbopogon citratus
MEMPPQHATAAAAVGQDEQSGSSPPLSPASAELANARWNPTKEQVAVLEGLYEHGLRTPSAEQIRQITGRLREHGAIEGKNVFYWFQNHKARQRQREKQDSFAYFTRLLHRPPPLPVLPMPPATPYHHRRRVPSPAPAPAPVAAAIAMPPPPPPPPSPAACNNNNGGARVMYRNAFYMPAPQAPAANATYYYPPPPQQVTVMYQYPRMELGQDKTMMMTTAAAAQQQHYPPAAMFQQAAAAAPLFPAERVGPSRQTLELFPLQPTFVLRRDKGRAAAATGSGSASLTSASASTATASASVSEESEGMEMSGNSNGEEAPPALPFYDFFGLQSGGR

SEQ ID NO: 19 - protein, 332 aa, Schizachyrium condensatum
MEMPLSAAASAGQLDEEGGSSSPPLSPASAALANARWIPTKEQVAVLEGLYEHGLHNPSAEQIQQITGRLREHGAIEGKNVFYWFQNHKARQRQREKQDSFAYFARLLRRPPPLPMLSSLPPPVPPYRHGRVPSPVPAPAPAPVPIAMPPPPPPPSPAAACNNNNGGARVTMYRNAFYMPTPQAPPANAAFYYPPPQQQVTVMYQYPRMELGQDKTMMLTTAAAQQQHHPAAGMFQQVAAAPHFFPLANGGAGPAERVGPSRETLELFPLQPTFVLRRDKGRAEAGGSGSASLTSASTASASISEESEGLESGGEVPALPFYDFFGLQSGRR

SEQ ID NO: 20 - protein, 310 aa, Tripsacum zopilotense
METPQPQSGAAHGQDEGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHGSVPAPAPAAIAMPPPPPPPAACNDNNGGARVIYRNPFYVAAQQAPPANAAYYYPQQQQQQVTVMYQYPRMEVAQDKMITRAAAPHFPPANGAGQQERVGPSRETLQLFPLQPTFVLRHDKGRAAAGNDSLTSTATATATATATASASISEDSDGLESGSSAKGVEAPALPFYDFFGLQSGGR

SEQ ID NO: 21 - protein, 313 aa, Zea diploperennis
METPQQQSGAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMPPPPAACNDNGGARVIYRNPFYVAAPQAPPANAAYYYPQPQQQQQQQVTVMYQYPRMEVAGQDKMMTRAAAQQHNGAGQQPGRAGHPSRETLQLFPLQPTFVLRHDKGRAANGNNNNDSLTSTSTATATASASISEDSDGLESGSSGKGVEEAPALPFYDFFGLQSSGGR

SEQ ID NO: 22 - protein, 313 aa, Zea mays
METPQQQSAAAHGQQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPPPPAACNDNGGARVIYRNPFYVAAPQAPPANAAYYYPQPQQQQQQVTVMYQYPRMEVAGQDKTMTRAAAPQQQQQHNGAGQQPGRAGHPSRETLQLFPLQPTFVLRHDKGRAANGSNNDSLTSTSTATATVTASASISEDSDGLESGSSGKGVEEAPALPFYDFFGLQSSGGR

SEQ ID NO: 23 - protein, 321 aa, Zea mays
METPQQQSGAAAVAHGQQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMPMPPPPAACNDNGGARVIYRNPFYVAAPQAPPANAAYYYPQPQQQQQQQQVTVMYQYPRMEVAGQDKMMSRAAAQQQQHNGAGQQPGRAGHPSRETLQLFPLQPTFVLRHDKGRAANGNNNNDSLTSTSTATATATASASISEDSDGLEIGSSGKGVEEAPALPFYDFFGLQSSGGR

SEQ ID NO: 24 - protein, 313 aa, Zea mays
METPQQQSAAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHGRVPAPPAMPMPMPPPPAACNDNGGARVIYRNPFYVAAPQAPPANAAYYYPQPQQQQQVTVMYQYPRMEVAGQDKMMTRAAAQQQQHNGAGQQPGRASRETLQLFPLQPTFVLRHDKGRAANGSNNDSLTSTSTATATATASASISEDSDGLESGSSGKGVEEAPALPFYDFFGLQSSGGR

SEQ ID NO: 25 - protein, 315 aa, Zea nicaraguensis
METPQQQSGAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHGRVPPPPAIPMPMPPPPAACNDNGGARVIYRNPFYVAAPQAPPANAAYYYPQPQQQVTVMYQYPRMEVAGQDKTMTRAAAPQQQQQHNGAGQQPGRAGHPSRETLQLFPLQPTFVLRHDKGRAANGNNNNDSQTSTSTATATATASISEDSDGLESGSSGKGVEEAPALPFYDFFGLQSSGGR

SEQ ID NO: 26 - protein, 338 aa, Andropogon chinensis
MAANVGAGRSVGGGGTAAGSGGVATTAVCRPSGSRWAPTPEQIRILKELYYGCGIRSPNSEQIQRITAMLRQHGKIEGKNVFYWFQNHKARERQKRRLTSLDVNVPAAAAGAADASSHLGVLSLSSPSGAAPPSPNLGFYAGNGGAGSTVLLDTSSDWGGSAATATETCFLQDYMGVMGGIGTGSAGAASPWACFSTSDPRAAATARAPTVTRAPETLPLFPIPIGGDDNQPRRPRPRHGVPVPVPLPADEAIPGGGSSSRYLPFWGAAPATASATSVGIIQQQSQLLQMQDQYYSFYSNTTQLPGTGNQDASSSAAAAASLELSLSSWCSPYPAGTM

SEQ ID NO: 27 - protein, 329 aa, Cymbopogon citratus
GGGGAGGIGAAAGSGSVAVCRPSGSRWTPTAEQIRILRELYYGCGIRSPNSEQIQRITAMLRQHGKIEGKNVFYWFQNHKARERQKRRLTNLDVNVPAADASTHLGVLSLSSPSSGAAPASPNLGFYAGNGGGAGSTVLMDTSSDWGGSAAMATETCFLQDYMGVMGGTGSAAAASPWACFSSSDPRAAATAWAPTPVTRAPETLPLFPIGGGDNQPRRPRPRHGVPVPVPVPAGEAILGGGGSSSRYLPFWGAASTTASGTASATTSVGIQQQHQLLQMQEQYYSFYSNATTQMPGTGSQDASSAAAAAASLELSLSSWCSPYPAGTM

SEQ ID NO: 28 - skipped

SEQ ID NO: 29 - protein, 315 aa, Tripsacum zopilotense
MAGNAGAGRSAGGGGAGGGSGSVAAPAVCRPSGSRWTPTPEQIRILKELYYGCGIRSPSSEQIQRITAMLRQHGKIEGKNVFYWFQNHKARERQKRRLTNNLDVNVPADATSQLGVLSLSSPSGAAPPSPTLGFFAGNGGASAVPLDTSSDWGSRGAAMATETCFLQDYMGVMATGSASQWPCFSSSDTMAAAAARAPETLPLFPTCDDDDDSQPPPRPRHGVPVPAGEAIRGGGSSSYLPFWGAASTTSTAGATSVAIQQQHQLLQLQEQYSFYSNTNQLAGTGSQDVSSAAAAAAALELSLSSWCSPYPAGTM

SEQ ID NO: 30 - protein, 325 aa, Zea diploperennis
MAANAGGGGSGGGSGSGSVAAPAVCRPSGSRWTPTPEQIRMLKELYYGCGIRSPSSEQIQRITAMLRQHGKIEGKNVFYWFQNHKARERQKRRLTNLDVNVPAAGAADAATSQLGVLSLSSPPSGAAPPSPTLGFYAAGNGGGSAVVLLDTSSDWGSSGAAMATETCFIQDYMGVTDTGSDAQWPCFSSSESDTIMAAAAARAATTRAPETLPLFPTCGDDDDDSQPPPRPRHAVPVPAGEPIRGGGGGSSSYLPFWGAASTTAGATSSVAIQQQHQLQEQYSFYSNSTQLAGTGSQDVSASAAAAALELSLSSWGSPYPAAGSM

SEQ ID NO: 31 - protein, 325 aa, Zea mays
MAGNAGAGRSAGGGGAGGGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSSTLGFYAAGNGGGSAVLLDTSSDWGSSGGAMATETCFLQDYMGVTDTGSSSQWPCFSSSDTIMAAAEARAPETLPLFPTCDDDDDSQPPPRPRHAVPVPAGETIRGGGSGSSSYYLPFWSAGAASTTAGATYSVTIQQQHQLQEQYSFYSNSTQLAGTGSQDVSASAAASAALELSLSSWCSPYPAAGSM

SEQ ID NO: 32 - protein, 302 aa, Zea mays
MAANAGGGGAGGGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQVGLHGRDGHGQLVAVATLLVVGHDNGGGRGAGGDDAGARDAPSLPDLRRRRRQRXQQLLAVLGCRVHNCRRHFFRCDAAATPAAGAVQLLQQQQQHPAGRHRQPRRIGFSSGGGRPRAEPQLMVLSLPCCREHV

SEQ ID NO: 33 - protein, 320 aa, Zea mays
MAANAGGGGAGGGSGSVVAQAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAVLLDTSSDWGSSGAAMATETCFLQDYMGVTDTGSSSQWPCFSSSESDTIMAAAATRAPETLPLFPTCGEDDDDDSQPPPRPRHAVPVPAGETIRGGGSSYLPFWGAASTTAGATSSVAIAIQQQHQLQEQYSFYSNSNSTQLAGTGSQDVSATAAAAAALELSLSSWCSPYPAAGSM

SEQ ID NO: 34 - protein, 312 aa, Zea nicaraguensis
MAANAGGGGAGGGSVAAPAVCRPSGSRWTPTPEQIRMLKELYYGCGIRSPSSEQIQRITAMLRQHGKIEGKNVFYWFQNHKARERQKRRLTNLDVNVPAAGAADATSQLGVLSLSSPPSGAAPPSPTLGFYAAGNGGGSAVVLLDTSSDWGSSGAAMATETCFLQDYMGVTDTGSSSQWPCFSSSDTIMAAATTRAPETLPLFPTCGGDDDDDSQPPPRPPHAVPFPAGETIRGGGGSYLPFWGAASTTAGATSSVAMQQQHQLQEQYSFYSNSTQLAGTGSQDVSASAAAAAALELSLSSWCSPYPAAGSM

SEQ ID NO: 35 - protein, 330 aa, Panicum hallii
MEAPQQGGGGALGLEGPGGAGAPLSPPLSPASAAAAALANSRWNPTKEQVAVLEGLYEHGLRTPSAEQIKQITARLREHGHIEGKNVFYWFQNHKARQRQKQKQDSFAYFTRLLRRPPPLPVLVSPPGPPYPHGRLPVPAAPAMTMAPSPAAAAAAAAAACNTSNGGTHVMYRAPFYMAAPQAPAANAAYYHTVQEQQQQQPWPVMYPRMAVAQDKMIPAAAMQHQQPPSHPGGAAYQAAAAPGNAPLHVVHLPAADAGGPSRETLQLFPLQPTFLLPDKGRPPTAAGTPASTASASFSGESESPGSPDSNGDAPAVPFYDFFGLQSGGR

SEQ ID NO: 36 - DNA, 993 bp, Panicum hallii
  source 1..993: mol_type = other DNA
atggaggcgccacagcaaggcggcggcggcgccctcggcctggagggccccggcggcgcgggcgcgccgctctcgccgccgctgtccccggcctccgccgcggcggcggcgctggcgaactcgaggtggaacccgaccaaggagcaggtggcggtgctggaggggctgtacgagcacggcctgcgcacccccagcgcggagcagatcaagcagatcacggcccggctgcgggagcacggccacatcgagggcaagaacgtcttctactggttccagaaccacaaggcccgccagcgccagaagcagaagcaggacagcttcgcctacttcaccaggctcctccgccgcccgccgccgctgcccgttctcgtcagcccccccggcccgccgtaccctcacggccgcctccccgtcccggccgcgccggcgatgacgatggccccctcacccgcggccgccgccgccgccgccgccgctgcatgcaacaccagcaacggtggcacgcatgtgatgtacagggctccattctacatggcagctccgcaggcgcccgcggcgaacgctgcctactaccacacggtgcaggagcagcagcagcagcagccgtggccagtcatgtacccgaggatggcggtcgcccaggacaagatgatccctgccgcggcgatgcagcaccagcagccgccgagccacccgggcggcgccgcgtaccaggccgccgcggcaccgggcaacgccccgctgcacgtggtccacctcccggcggcggacgccgggggccccagccgcgagacgctgcagctgttcccgctgcagcccaccttcctgctgcccgacaaagggcgcccccccaccgccgccggcacgccggcgtcgacggcgtccgcttcgttctcgggggagtccgagagcccggggagcccggactcgaacggcgacgcgcccgcggtgcccttctacgacttcttcggcctccagtccggaggccgctga

SEQ ID NO: 37 - protein, 322 aa, Miscanthus lutarioriparius
MEMPQQQAAANAAQDEGGSPPLSPAAAAAAALANARWNPTKEQVAVLEGLYEHGLRTPSAEQIQQITGRLREHGAIEGKNVFYWFQNHKARQRQRQKQDSFAYFTRLLRRPPPLPVLSMPPAPPYHHGRVPSSAPAPAPPAIAMPPPPPPPSPAACNNNNGGARVMYRNAFYMPAPQAPPANAAYYYPPPQQQVTVMYQYQDKMTAAAAQQQHHYPAAMFQQAAAAPHFPSANGGSGPAERVGPRRETLELFPLQPTFVLRRDKGRAAAAGGSASALTSASTATASASVSEESEGLESANCNGEEEPALPFYDFFGLQSGGR

SEQ ID NO: 38 - DNA, 969 bp, Miscanthus lutarioriparius
  source 1..969: mol_type = other DNA
atggagatgccgcagcagcaagccgccgccaacgccgcccaggacgagggcgggtcgccgccgctgtccccggcggccgccgcggcggccgcgctggcgaacgcgcggtggaacccgaccaaggagcaggtggcggtgctggaggggctgtacgagcacggcctgcgcaccccgagcgcggagcagatacagcagatcacgggcaggctgcgggagcacggcgccatcgagggcaagaacgtcttctactggttccagaaccacaaggcccgccaacgccagcgccagaagcaggacagcttcgcctacttcaccaggctcctccgccgtcccccgccgctgcccgtgctctccatgccgcccgcgccgccgtaccatcacggccgcgtcccgtcgtctgcgccggcgccggcgccgcccgcgatcgcgatgccgccgccgcctcctcctccgtcgcccgctgcatgcaacaacaacaacggcggcgcgcgtgtgatgtacaggaatgcattctacatgcctgcgccgcaggcgccccctgcaaatgctgcctactactacccgccgccgcagcagcaggtgacagtcatgtaccagtaccaggacaagatgaccgcggccgcggcgcagcagcagcaccactacccggccgccatgttccagcaggccgccgcagcaccgcacttcccgtctgccaacggcggctccgggccagcggaacgcgtcggcccccgccgcgagacgctcgagctgttcccgctccagcccaccttcgtgctgcggcgcgacaaggggcgcgctgcagccgctgggggcagcgcctccgccctgacgtcggcgtcgacggcgacggcgtccgcttccgtctccgaggagtcggagggcctggagagcgccaactgcaacggcgaggaggagcccgcgctgccgttctatgacttcttcggtctccagtccggaggccgctga

(19) United States
(12) Patent Application Publication
(10) Pub. No.: US 2025/0197879 A1
(43) Pub. Date: Jun. 19, 2025

(54) MORPHOREGULATORS FOR REGENERATION OF MAIZE SOMATIC EMBRYOS

(71) Applicant: INARI AGRICULTURE TECHNOLOGY, INC., Cambridge, MA (US)

(72) Inventors: ZHANYUAN JON ZHANG, West Lafayette, IN (US); Wenbin Mei, Lexington, MA (US); JEFFREY R. BERINGER, Carmel, IN (US); MANMEET SINGH, West Lafayette, IN (US); RHODA ARABA TAWIAH BREW-APPIAH, West Lafayette, IN (US)

(21) Appl. No.: 18/847,439

(22) PCT Filed: Mar. 17, 2023
(86) PCT No.: PCT/US2023/064616

§ 371 Date: Sep. 16, 2024

Related U.S. Application Data
(60) Provisional application No. 63/269,590, filed on Mar. 18, 2022.
(60) Provisional application No. 63/371,818, filed on Aug. 18, 2022.

(51) Int. Cl. C12N 15/82; C07K 14/415; C12N 9/22; C12N 15/11

(52) U.S. Cl. CPC C12N 15/8262; C07K 14/415; C12N 9/22; C12N 15/111; C12N 15/8213; C12N 2310/20

(57) ABSTRACT

WUSCHEL and WUSCHEL-related homeobox polypeptides that can be used to improve the capacity of the plant cells to regenerate embryogenic plant tissues, plant organs, and whole plants are disclosed. Also disclosed are plant cells comprising the polypeptides and related methods for improving the capacity of the plant cells to regenerate embryogenic plant tissues, plant organs, and whole plants.

### CROSS-REFERENCE TO RELATED APPLICATIONS

This application claims priority to provisional applications U.S. Ser. No. 63/269,590 filed Mar. 18, 2022 and U.S. Ser. No. 63/371,818 filed Aug. 18, 2022, which are incorporated herein by reference in their entireties.

### SEQUENCE LISTING XML

The instant application contains a sequence listing, which has been submitted in XML file format by electronic submission and is hereby incorporated by reference in its entirety. The XML file, created on Mar. 14, 2023, is named P13785WO00.xml and is 51,903 bytes in size.

### BACKGROUND

To overcome recalcitrance of corn to genetic transformation, transgenes encoding morphoregulators BABYBOOM (ODP2) and/or WUSCHEL2 (WUS2) have been transiently expressed in maize cells to stimulate somatic embryogenesis (Lowe et al. 2016, Lowe et al. 2018). Such somatic embryogenesis promotes formation of direct embryogenic callus from which new shoots can be generated, making some previously recalcitrant tissues accessible to transformation. Although embryogenic enhancers have been tried and work especially in certain combinations (e.g. ZmBBM2 and ZmWUS2), they often have limited effectiveness on some lines or cytotoxicity that requires sophisticated deployment. A need exists for additional reagents to expand the range of germplasm accessible for transformation.

### SUMMARY

Methods of producing a regenerable plant structure, comprising introducing a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37 in a maize plant cell; and culturing the maize plant cell to produce the regenerable plant structure are provided.

Maize plant cells comprising a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37, wherein expression of the polypeptide increases proliferation, somatic embryogenesis, and/or regeneration capacity of the maize plant cell are provided. Methods for producing a maize plant comprising regenerating a maize plant from the maize plant cells are provided.

Recombinant polynucleotides comprising (a) a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37, or (b) a polynucleotide having at least 90%, at least 95%, at least 96%, at least 97%, at least 98%, at least 99%, or at least 99.5% sequence identity across the entire length of SEQ ID NO: 7, 8, 9, 36, or 38; wherein the polynucleotide of (a) or (b) is operably linked to a heterologous promoter functional in a plant cell are provided. Also provided are vectors comprising the recombinant polynucleotides.

### BRIEF DESCRIPTION OF THE DRAWINGS

FIG. 1A-B shows pIN3261 (FIG. 1A) and pIN3262 (FIG. 1B) calli before the selection 1 step.

FIG. 2A-D shows pIN3258 (FIG. 1A), pIN3259 (FIG. 1B), pIN3260 (FIG. 1C), and pIN3239 empty control (FIG. 1D) calli below before selection 1 stage.

FIG. 3A-D shows photos of somatic embryogenesis responses at the end of Rest stage for pIN3258 (FIG. 3A), pIN3261 (FIG. 3B), pIN3239 (FIG. 3C), and pIN2541 (FIG. 3D).

FIG. 4A shows the good quality of somatic embryos as induced by pIN3267 (top) and the high percentage of immature embryos producing somatic embryos (bottom), i.e., embryo induction frequency. FIG. 4B shows few somatic embryos as induced by pIN3268 (top) and the very low percentage of immature embryos producing somatic embryos (bottom). FIG. 4C shows the very low percentage of immature embryos producing somatic embryos as induced by pIN3239.

FIG. 5A shows the multiple and good quality of somatic embryos (top) as well as the high percentage of immature embryos producing somatic embryos (bottom) as induced by pIN3956 containing PhWOX2.2 cassette. FIG. 5B shows the few and poor quality of somatic embryos (top) as well as the very low percentage of immature embryos producing somatic embryos (bottom) as induced by empty control construct pIN3768 carrying PAT-mScarlet cassettes only. FIG. 5C shows the statistics of embryo productivity.

FIG. 6 shows regeneration response of three editing constructs carrying MlWUS2 or SbWOX5 at the end of Regeneration medium (ZM9177), showing many fold increase in shoot regeneration by pIN3759 as compared with pIN3760 and pIN3761. pIN3760 carrying SbWOX5 also produced more shoots than pIN3761 empty control. Recalcitrant maize inbred GIDA8989 was transformed. A, B, and C were three maize ears used as replicates in this experiment.

### DETAILED DESCRIPTION

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art. Where a term is provided in the singular, the inventors also contemplate embodiments described by the plural of that term.

The phrase “allelic variant” as used herein refers to a polynucleotide or polypeptide sequence variant that occurs in a different strain, variety, or isolate of a given organism.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “embryo productivity” refers to an assigned number that reflects how many somatic embryos develop on each immature embryo that is embryogenic, wherein the assigned number is determined by a subjective somatic embryogenesis score from 0 to 4 and wherein a higher score indicates an increase in somatic embryo production.

The term “genome” refers to the entire complement of genetic material (genes and non-coding sequences) that is present in an organism or cell, including both nuclear and organellar DNA.

As used herein. “heterologous” refers to a polynucleotide or peptide sequence located in, e.g., a genome or a vector, in a context other than that in which the sequence occurs in nature. For example, a promoter that is operably linked to a gene other than the gene that the promoter is operably linked to in nature is a heterologous promoter.

The phrase “improved plant cell regenerative potential” as used herein refers to the ability of a given plant cell to form a somatic embryo, embryogenic callus, a somatic meristem, organogenic callus, a shoot, or a shoot further comprising roots in comparison to a control plant cell.

As used herein, the terms “include,” “includes,” and “including” are to be construed as at least having the features to which they refer while not excluding any additional unspecified features.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the phrase “plant cell” can refer either a plant cell having a plant cell wall or to a plant cell protoplast lacking a plant cell wall.

As used herein, the phrase “somatic embryo induction frequency” refers to the percentage of immature embryos producing somatic embryos.

As used herein, the phrase “target gene” can refer to a gene located in the genome that is to be modified by genome editing systems provided herein. Embodiments of target genes include (protein-)coding sequence, non-coding sequence, and combinations of coding and non-coding sequences. Target gene edits include nucleotide substitutions, insertions, and/or deletions in one or more elements of a gene that include a transcriptional enhancer or promoter, a 5′ or 3′ untranslated region, a mature or precursor RNA coding sequence, an intron, a splice donor and/or acceptor, a protein coding sequence, a polyadenylation site, and/or a transcriptional terminator.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Plant cells and related systems, methods, and compositions that provide for improved plant cell regenerative potential in comparison to control plant cells are provided herein. In certain embodiments, improved plant cell regenerative potential is provided by introducing a polynucleotide encoding a WUSCHEL or WUSCHEL-related homeobox (WUS/WOX) polypeptide in the plant cell. In certain embodiments, the WUS/WOX polypeptide comprises a Sorghum bicolor WOX5 (SbWOX5) polypeptide of SEQ ID NO: 1 or an allelic variant thereof.

In certain embodiments, the WUS/WOX polypeptide comprises a Miscanthus lutarioriparius WUS2 (MlWUS2) polypeptide of SEQ ID NO: 2 or an allelic variant thereof. In certain embodiments, the WUS/WOX polypeptide comprises a Setaria italica WOX4 (SiWOX4) polypeptide of SEQ ID NO: 3 or an allelic variant thereof. In certain embodiments, the WUS/WOX polypeptide comprises a Panicum hallii WOX2.2 (PhWOX2.2) polypeptide of SEQ ID NO: 35 or an allelic variant thereof. In certain embodiments, the WUS/WOX polypeptide comprises a Miscanthus lutarioriparius WOX2.2 (MlWOX2.2) polypeptide of SEQ ID NO: 37 or an allelic variant thereof. Homologs of SbWOX5/SiWOX4 and MlWUS2 from related grass species are also provided. Such homologs of SbWOX5/SiWOX4 include, for example, the polypeptides of SEQ ID NOs: 17, 18, 19, 20, 21, 22, 23, 24, and 25. Such homologs of MlWUS2 include, for example, the polypeptides of SEQ ID NOs: 26, 27, 29, 30, 31, 32, 33, and 34. In certain embodiments, SbWOX5, MlWUS2, SiWOX4, PhWOX2.2, and MlWOX2.2 polypeptides, homologs thereof, variants thereof, and allelic variants thereof can significantly increase somatic embryogenesis from callus of otherwise recalcitrant germplasm. In certain embodiments, the polynucleotide encodes a WUS/WOX polypeptide comprising an amino acid sequence having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37. In certain embodiments, the allelic variant of SEQ ID NO: 1, 2, 3, 17, 18, 19, 20, 21, 22, 23, 24, 25, 26, 27, 29, 30, 31, 32, 33, 34, 35, or 37 comprises a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37. In certain embodiments, the polynucleotide encoding the WUS/WOX polypeptide has at least 90%, at least 95%, at least 96%, at least 97%, at least 98%, at least 99%, or at least 99.5% sequence identity across the entire length of SEQ ID NO: 7, 8, 9, 36, or 38. In certain embodiments, the polynucleotide encoding the WUS/WOX polypeptide has at least 90%, at least 95%, at least 96%, at least 97%, at least 98%, at least 99%, or at least 99.5% sequence identity across the entire length of SEQ ID NO: 7, 8, 9, 36, or 38. In certain embodiments, an aforementioned WUS polypeptide can comprise a homeodomain, an acidic region, WUS box motif (e.g., SEQ ID NO: 13) and/or an EAR-like (for ERF-associated amphiphilic repression) motif (e.g., SEQ ID NO: 14). In certain embodiments, an aforementioned WOX polypeptide can comprise a homeodomain, an acidic region, and a WUS-Like motif (e.g., SEQ ID NO: 15 or 16). In certain embodiments, a polynucleotide encoding a BABY BOOM (BBM) polypeptide having at least 90%, at least 95%, at least 96%, at least 97%, at least 98%, at least 99%, or at least 99.5% sequence identity across the entire length of SEQ ID NO: 5 or 6 is also introduced in the plant cell with an aforementioned WUS/WOX polypeptide.

In certain embodiments, the polynucleotide encoding the WUS/WOX polypeptide is introduced in isolated plant cells or plant protoplasts (i.e., are not located in undissociated or intact plant tissues, plant parts, or whole plants). In certain embodiments, the plant cells are obtained from any plant part or tissue or callus. In certain embodiments, the culture includes plant cells obtained from a plant tissue, a cultured plant tissue explant, whole plant, intact nodal bud, shoot apex or shoot apical meristem, root apex or root apical meristem, lateral meristem, intercalary meristem, seedling, whole seed, halved seed or other seed fragment, zygotic embryo, somatic embryo, immature embryo, ovule, pollen, microspore, anther, hypocotyl, cotyledon, leaf, petiole, stem, tuber, root, callus, or plant cell suspension. In certain embodiments, the plant cell is derived from the L1 or L2 layer of an immature or mature embryo of a monocot plant (e.g., maize, wheat, sorghum, or rice).

In certain embodiments, the polynucleotide encoding the WUS/WOX polypeptide is introduced in plant cells that are located in undissociated or intact plant tissues, plant parts, plant explants, or whole plants. In certain embodiments, the plant cell can be located in an intact nodal bud, a cultured plant tissue explant, shoot apex or shoot apical meristem, root apex or root apical meristem, lateral meristem, intercalary meristem, seedling, whole seed, halved seed or other seed fragment, zygotic embryo, somatic embryo, immature embryo, ovule, pollen, microspore, anther, hypocotyl, cotyledon, leaf, petiole, stem, tuber, root, or callus. In certain embodiments, the explants used include immature embryos. Immature embryos (e.g., immature maize embryos) include 1.8-2.2 mm embryos, 1-7 mm embryos, and 3-7 mm embryos. In certain embodiments, the aforementioned embryos are obtained from mature ear-derived seed, leaf bases, leaves from mature plants, leaf tips, immature inflorescences, tassels, immature ears, and silks. In various aspects, the plant-derived explant includes immature embryos, 1.8-2.2 mm embryos, 1-7 mm embryos, and 3.5-7 mm embryos. In an aspect, the embryos used in the disclosed methods can be derived from mature ear-derived seed, leaf bases, leaves from mature plants, leaf tips, immature inflorescences, tassel, immature ear, or silks. In certain embodiments, the plant cell is a pluripotent plant cell (e.g., a stem cell or meristem cell). In certain embodiments, the plant cell is located within the L1 or L2 layer of an immature or mature embryo of a monocot plant (e.g., maize, wheat, sorghum, or rice).

In certain embodiments, the plant cells where the polynucleotide encoding the WUS/WOX polypeptide is introduced, as well as the related methods, systems, or compositions provided herein can include plant cells obtained from or located in any monocot plant species of interest, for example, row crop plants and turf grasses. In certain non-limiting embodiments, the plant cells are obtained from or located in barley (Hordeum vulgare), maize (Zea mays L.), millets (Setaria spp, Echinochloa spp, Eleusine spp, Panicum spp., Pennisetum spp.), oats (Avena sativa), oil palm (Ellis quineensis), rice (Oryza sativa L.), rye (Secale cereale), sorghum (Sorghum bicolor), sugarcanes (Saccharum spp.) or wheat (Tritium aestivum). In certain embodiments, the plant cell is a maize plant cell.

In certain embodiments, the plant cells can comprise haploid, diploid, or polyploid plant cells or plant protoplasts, for example, those obtained from a haploid, diploid, or polyploid plant, plant part or tissue, or callus. In certain embodiments, plant cells in culture (or the regenerated plant, progeny seed, and progeny plant) are haploid or can be induced to become haploid; techniques for making and using haploid plants and plant cells are known in the art, see, e.g., methods for generating haploids in Arabidopsis thaliana by crossing of a wild-type strain to a haploid-inducing strain that expresses altered forms of the centromere-specific histone CENH3, as described by Maruthachalam and Chan in “How to make haploid Arabidopsis thaliana”, protocol available at www[dot]openwetware[dot]org/images/d/d3/Haploid_Arabidopsis_protocol[dot]pdf; (Ravi et al. (2014) Nature Communications, 5:5334, doi: 10.1038/ncomms6334). Haploids can also be obtained in a wide variety of monocot plants (e.g., maize, wheat, rice, sorghum, barley) by crossing a plant comprising a mutated CENH3 gene with a wildtype diploid plant to generate haploid progeny as disclosed in U.S. Pat. No. 9,215,849, which is incorporated herein by reference in its entirety. Haploid-inducing maize lines that can be used to obtain haploid maize plants and/or cells include Stock 6, MHI (Moldovian Haploid Inducer), indeterminate gametophyte (ig) mutation, KEMS, RWK, ZEM, ZMS, KMS, and well as transgenic haploid inducer lines disclosed in U.S. Pat. No. 9,677,082, which is incorporated herein by reference in its entirety. Examples of haploid cells include but are not limited to plant cells obtained from haploid plants and plant cells obtained from reproductive tissues, e.g., from flowers, developing flowers or flower buds, ovaries, ovules, megaspores, anthers, pollen, megagametophyte, and microspores. In certain embodiments where the plant cell or plant protoplast is haploid, the genetic complement can be doubled by chromosome doubling (e.g., by spontaneous chromosomal doubling by meiotic non-reduction, or by using a chromosome doubling agent such as colchicine, oryzalin, trifluralin, pronamide, nitrous oxide gas, anti-microtubule herbicides, anti-microtubule agents, and mitotic inhibitors) in the plant cell or plant protoplast to produce a doubled haploid plant cell or plant protoplast wherein the complement of genes or alleles is homozygous; yet other embodiments include regeneration of a doubled haploid plant from the doubled haploid plant cell or plant protoplast. Another embodiment is related to a hybrid plant having at least one parent plant that is a doubled haploid plant provided by this approach. Production of doubled haploid plants provides homozygosity in one generation, instead of requiring several generations of self-crossing to obtain homozygous plants. The use of doubled haploids is advantageous in any situation where there is a desire to establish genetic purity (i.e. homozygosity) in the least possible time. Doubled haploid production can be particularly advantageous in slow-growing plants, such as fruit and other trees, or for producing hybrid plants that are offspring of at least one doubled-haploid plant.

In certain embodiments, the plant cells where the polynucleotide encoding the WUS/WOX polypeptide is introduced can be plant cells that are (a) encapsulated or enclosed in or attached to a polymer (e.g., pectin, agarose, or other polysaccharide) or other support (solid or semi-solid surfaces or matrices, or particles or nanoparticles); (b) encapsulated or enclosed in or attached to a vesicle or liposome or other fluid compartment; or (c) not encapsulated or enclosed or attached. In certain embodiments, the plant cells can be in liquid or suspension culture, or cultured in or on semi-solid or solid media, or in a combination of liquid and solid or semi-solid media (e.g., plant cells or protoplasts cultured on solid medium with a liquid medium overlay, or plant cells or protoplasts attached to solid beads or a matrix and grown with a liquid medium). In certain embodiments, the plant cells encapsulated in a polymer (e.g., pectin, agarose, or other polysaccharide) or other encapsulating material, enclosed in a vesicle or liposome, suspended in a mixed-phase medium (such as an emulsion or reverse emulsion), or embedded in or attached to a matrix or other solid support (e.g., beads or microbeads, membranes, or solid surfaces).

In a related embodiment, the disclosure provides arrangements of plant cells having improved plant cell regenerative potential in the systems, methods, and compositions described herein, such as arrangements of plant cells convenient for screening purposes or for high-throughput and/or multiplex transformation or gene editing experiments. In an embodiment, the disclosure provides an arrangement of multiple plant cells comprising: (a) a polynucleotide encoding a WUS/WOX polypeptide; and optionally (b) a genome editing system. In another embodiment, the disclosure provides an array including a plurality of containers, each including at least one plant cell or plant protoplast having improved plant cell regenerative potential. In an embodiment, the disclosure provides arrangements of plant cells having the polynucleotide encoding the WUS/WOX polypeptide and optionally the genome editing system, wherein the plant cells are in an arrayed format, for example, in multi-well plates, encapsulated or enclosed in vesicles, liposomes, or droplets (useful, (e.g., in a microfluidics device), or attached discretely to a matrix or to discrete particles or beads; a specific embodiment is such an arrangement of multiple plant cells having improved plant cell regenerative potential provided in an arrayed format, further including a genome editing system (e.g., an RNA-guided nuclease, at least one guide RNA), which may be different for at least some locations on the array or even for each location on the array, and optionally at least one chemical, enzymatic, or physical delivery agent.

In the systems and methods provided herein, the polynucleotide encoding the WUS/WOX polypeptide and a genome editing system can be introduced in the plant cell in any temporal order. In certain embodiments, the genome editing system and the polynucleotide encoding the WUS/WOX polypeptide are introduced simultaneously. In other embodiments, the genome editing system is introduced after the polynucleotide encoding the WUS/WOX polypeptide. In other embodiments, the genome editing system is introduced before the polynucleotide encoding the WUS/WOX polypeptide. In summary, the genome editing system can be provided to a plant cell either previous to, concurrently with, or subsequent to introducing the polynucleotide encoding the WUS/WOX polypeptide to the plant cell.

Plant cells having improved plant cell regenerative potential conferred by expression of a WUS/WOX polypeptide are provided herein. Also provided by the disclosure are compositions derived from or grown from the plant cell or plant protoplast having improved plant cell regenerative potential, provided by the systems and methods disclosed herein; such compositions include multiple protoplasts or cells, callus, a somatic embryo, a somatic meristem, embryogenic callus, or a regenerated plant grown from the plant cell or plant protoplast having improved plant cell regenerative potential. Improved plant cell regenerative potential in plant cells can be assessed by a variety of techniques. In certain embodiments, such techniques can compare the numbers and/or amount of regenerable plant structures (e.g., immature embryos, somatic embryos, embryogenic calli, somatic meristems, organogenic calli, shoots, or shoots further comprising roots) formed and/or recovered from a given number of plant cells comprising the polynucleotide encoding the WUS/WOX polypeptide versus control plant cells without the polynucleotide. In certain embodiments, expression of the WUS/WOX polypeptide results in an increased somatic embryo induction frequency or embryo productivity relative to a control plant cell lacking the polynucleotide encoding the polypeptide. In certain embodiments, the somatic embryo induction frequency is at least about 50%, at least about 60%, at least about 70%, at least about 80%, at least about 90%, or at least about 95%. In certain embodiments, somatic embryo induction frequency is increased at least 2-fold, at least 5-fold, at least 10-fold, or at least 20-fold relative to a control maize plant cell lacking the polynucleotide encoding the polypeptide. In certain embodiments, the embryo productivity is at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, or at least about 2.5. In certain embodiments, the embryo productivity is from about 1 to about 3 or from about 1.5 to about 2.5.

When both somatic embryo induction frequency and embryo productivity are high, the transformation rate is high and the culture is robust.

In certain embodiments, an attribute of tissues selected for introduction of the WUS/WOX polynucleotides can be the presence of dividing cells and the ability to grow in tissue culture media. These tissues include, but are not limited to, dividing cells from young maize leaf, meristems and scutellar tissue from about 8 or 10 to about 12 or 14 days after pollination (DAP) embryos. The isolation of maize embryos has been described in several publications (Brettschneider, Becker, and L6rz 1997; Leduc et al. 1996; Frame et al. 2011; K. Wang and Frame 2009). In certain embodiments, basal leaf tissues (e.g., leaf tissues located about 0 to 3 cm from the ligule of a maize plant; Kirienko, Luo, and Sylvester 2012) are selected for introduction of the polynucleotides. In certain embodiments, such increases in numbers and/or amounts of regenerable plant structures can be observed in about 1, 2, or 3 to about 7, 10, 14, 30, or 60 days following the introduction of a polynucleotide encoding the WUS/WOX polypeptide. Methods for obtaining regenerable plant structures and regenerating plants from the plant cells provided herein can be adapted from methods disclosed in US Patent Application Publication No. 20170121722, which is incorporated herein by reference in its entirety and specifically with respect to such disclosure. In certain embodiments, single plant cells subjected to the introduction of the polynucleotide will give rise to single regenerable plant structures. In certain embodiments, the single regenerable plant cell structure can form from a single cell on, or within, an explant that has been subjected to the introduction of the polynucleotide and optionally subjected to treatment with a genome editing system. In certain embodiments, initiation or formation of the single plant cell regenerable structure can occur where single-cell-derived cell or tissue proliferation (e.g., growth of callus, non-differentiated callus, embryogenic callus and organogenic callus) occurring before initiation of the regenerable plant structure is reduced or absent. In certain embodiments, regenerable plant structures from plant cells subjected to the introduction of the polynucleotide and optionally a genome editing system can be form the regenerable plant structure in the absence of exogenous cytokinin or with levels of cytokinin that are lower than those required to initiate formation of the regenerable structure from a control cell. In certain embodiments, regenerable plant structures from plant cells subjected to the introduction of the polynucleotide and optionally a genome editing system can be identified and/or selected via a positive growth selection based on the ability of those plant cells to initiate and/or form the regenerable plant structures more rapidly than adjacent plant cells that have not been subjected to the introduction of the polynucleotide. In certain embodiments, such positive growth selection can obviate or reduce the need to use a traditional negative selection system where an antibiotic or herbicide is used to inhibit growth of adjacent, non-transformed cells that do not contain a gene that confers resistance to the antibiotic or herbicide. Nonetheless, embodiments where a selectable marker gene conferring resistance to an antibiotic, herbicide, or other agent can be introduced into the plant cell at least temporarily during initiation and/or formation of the regenerable plant cell structures to facilitate identification and recovery.

In some embodiments, methods provided herein can include the additional step of growing or regenerating a plant from a plant cell comprising the polynucleotide encoding the WUS/WOX polypeptide or from a regenerable plant structure obtained from that plant cell. In certain embodiments, the plant can further comprise an inserted transgene, a target gene edit, or genome edit as provided by the methods and compositions disclosed herein. In certain embodiments, callus is produced from the plant cell, and plantlets and plants produced from such callus. In other embodiments, whole seedlings or plants are grown directly from the plant cell without a callus stage. Thus, additional related aspects are directed to whole seedlings and plants grown or regenerated from the plant cell or plant protoplast having a target gene edit or genome edit, as well as the seeds of such plants. In certain embodiments wherein the plant cell or plant protoplast is subjected to genetic or epigenetic modification (for example, stable or transient expression of a transgene, gene silencing, epigenetic silencing, or genome editing by means of, e.g., an RNA-guided nuclease), the grown or regenerated plant exhibits a phenotype associated with the genetic or epigenetic modification. In certain embodiments, the grown or regenerated plant includes in its genome two or more genetic or epigenetic modifications that in combination provide at least one phenotype of interest. In certain embodiments, a heterogeneous population of plant cells having a target gene edit or genome edit, at least some of which include at least one genetic or epigenetic modification, is provided by the method; related aspects include a plant having a phenotype of interest associated with the genetic or epigenetic modification, provided by either regeneration of a plant having the phenotype of interest from a plant cell or plant protoplast selected from the heterogeneous population of plant cells having a target gene or genome edit, or by selection of a plant having the phenotype of interest from a heterogeneous population of plants grown or regenerated from the population of plant cells having a target gene edit or genome edit. Examples of phenotypes of interest include herbicide resistance, improved tolerance of abiotic stress (e.g., tolerance of temperature extremes, drought, or salt) or biotic stress (e.g., resistance to nematode, bacterial, or fungal pathogens), improved utilization of nutrients or water, modified lipid, carbohydrate, or protein composition, improved flavor or appearance, improved storage characteristics (e.g., resistance to bruising, browning, or softening), increased yield, altered morphology (e.g., floral architecture or color, plant height, branching, root structure). In an embodiment, a heterogeneous population of plant cells having a target gene edit or genome edit (or seedlings or plants grown or regenerated therefrom) is exposed to conditions permitting expression of the phenotype of interest; e.g., selection for herbicide resistance can include exposing the population of plant cells having a target gene edit or genome edit (or seedlings or plants grown or regenerated therefrom) to an amount of herbicide or other substance that inhibits growth or is toxic, allowing identification and selection of those resistant plant cells (or seedlings or plants) that survive treatment. Methods for obtaining regenerable plant structures and regenerating plants from plant cells or regenerable plant structures can be adapted from published procedures (Roest and Gilissen, Acta Bot. Neerl., 1989, 38(1), 1-23; Bhaskaran and Smith, Crop Sci. 30(6):1328-1337; Ikeuchi et al., Development, 2016, 143: 1442-1451). Methods for obtaining regenerable plant structures and regenerating plants from plant cells or regenerable plant structures can also be adapted from US Patent Application Publication No. 20170121722, which is incorporated herein by reference in its entirety and specifically with respect to such disclosure. Also provided are heterogeneous populations, arrays, or libraries of such plants, succeeding generations or seeds of such plants grown or regenerated from the plant cells or plant protoplasts, having a target gene edit or genome edit, parts of the plants (including plant parts used in grafting as scions or rootstocks), or products (e.g., fruits or other edible plant parts, cleaned grains or seeds, edible oils, flours or starches, proteins, and other processed products) made from the plants or their seeds. Embodiments include plants grown or regenerated from the plant cells having a target gene edit or genome edit, wherein the plants contain cells or tissues that do not have a genetic or epigenetic modification, e.g., grafted plants in which the scion or rootstock contains a genetic or epigenetic modification, or chimeric plants in which some but not all cells or tissues contain a genetic or epigenetic modification. Grafted plants can be grafts between the same or different (generally related) species. Additional related aspects include a hybrid plant provided by crossing a first plant grown or regenerated from a plant cell or plant protoplast having a target gene edit or genome edit and having at least one genetic or epigenetic modification, with a second plant, wherein the hybrid plant contains the genetic or epigenetic modification; also contemplated is seed produced by the hybrid plant. Also envisioned as related aspects are progeny seed and progeny plants, including hybrid seed and hybrid plants, having the regenerated plant as a parent or ancestor. The plant cells and derivative plants and seeds disclosed herein can be used for various purposes useful to the consumer or grower. The intact plant itself may be desirable, e.g., plants grown as cover crops or as ornamentals. In other embodiments, processed products are made from the plant or its seeds, such as extracted proteins, oils, sugars, and starches, fermentation products, animal feed or human food, wood and wood products, pharmaceuticals, and various industrial products.

A WUS/WOX polypeptide can be provided to a cell (e.g., a plant cell or plant protoplast) by any suitable technique. In certain embodiments, the WUS/WOX polypeptide is provided by directly contacting a plant cell with the WUS/WOX polypeptide or the polynucleotide that encodes the WUS/WOX polypeptide. In certain embodiments, the WUS/WOX polypeptide is provided by transporting the WUS/WOX polypeptide or a polynucleotide that encodes WUS/WOX polypeptide into a plant cell or plant protoplast using a chemical, enzymatic, or physical agent. In certain embodiments, the WUS/WOX polypeptide is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of a plant cell or plant protoplast with a polynucleotide encoding the WUS/WOX polypeptide; see, e.g., Broothaerts et al. (2005) Nature, 433:629-633. In an embodiment, the WUS/WOX polypeptide is provided by transcription in a plant cell or plant protoplast of a DNA that encodes the polypeptide and is stably integrated in the genome of the plant cell or is provided to the plant cell or plant protoplast in the form of a plasmid or expression vector (e.g., a viral vector) that encodes the polypeptide. In certain embodiments, the polypeptide is provided to the plant cell or plant protoplast as a polynucleotide that encodes the polypeptide, e.g., in the form of an RNA (e.g., mRNA or RNA containing an internal ribosome entry site (IRES)) encoding the polypeptide. A genome editing system can also be introduced into the plant cells by similar techniques.

Transient expression of a WUS/WOX polypeptide can be achieved by a variety of techniques. Certain embodiments are useful in effectuating transient expression of the WUS/WOX polypeptide without remnants or selective genetic markers occurring in progeny. In certain embodiments, the WUS/WOX polypeptide is provided directly to the plant cells, systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, the WUS/WOX polypeptide is targeted to the plant cell or cell nucleus in a manner that ensures transient expression (e.g., by methods adapted from Gao et al. 2016; or Li et al. 2009). In certain embodiments, the WUS/WOX polypeptide is delivered into the plant cell by delivery of the polypeptide itself in the absence of any polynucleotide that encodes the polypeptide. In certain embodiments, the WUS/WOX protein can be produced in a heterologous system, purified and delivered to plant cells by particle bombardment (e.g., by methods adapted from Martin-Ortigosa and Wang 2014). In embodiments where the WUS/WOX polypeptide is delivered in the absence of any encoding polynucleotides, the delivered polypeptide is expected to degrade over time in the absence of ongoing expression from any introduced encoding polynucleotides to result in transient expression. In certain embodiments, the WUS/WOX polypeptide is delivered into the plant cell by delivery of a polynucleotide that encodes the polypeptide. In certain embodiments, the polypeptide can be encoded on a bacterial plasmid and delivered to plant tissue by particle bombardment (e.g., by methods adapted from Hamada et al. 2018; or Kirienko, Luo, and Sylvester 2012). In certain embodiments, the polypeptide can be encoded on a T-DNA and transiently transferred to plant cells using Agrobacterium (e.g., by methods adapted from Leonelli et al. 2016; or Wu et al. 2014). In certain embodiments, the polypeptide can be encoded in a viral genome and delivered to plants (e.g., by methods adapted from Honig et al. 2015). In certain embodiments, the polypeptide can be encoded in mRNA or an RNA comprising an IRES and delivered to target plant cells. In certain embodiments, the WUS/WOX polypeptide is delivered into the plant cell by delivery of a polynucleotide that encodes the polypeptide. In certain embodiments, the polynucleotide that encodes the WUS/WOX polypeptide is not integrated into a plant cell genome (e.g., as a polynucleotide lacking sequences that provide for integration, by agroinfiltration on an integration deficient T-DNA vector or system, or in a viral vector), is not operably linked to polynucleotides which provide for autonomous replication, and/or only provided with factors (e.g., viral replication proteins) that provide for autonomous replication. Suitable techniques for transient expression including biolistic and other delivery of polynucleotides, agroinfiltration, and use of viral vectors disclosed by Canto, 2016 and others can be adapted for transient expression of the polypeptides provided herein. In certain embodiments, the polynucleotide that encodes the WUS/WOX polypeptide is integrated into a plant cell genome (e.g., a nuclear or plastid genome) and transient expression of the polypeptide is effectuated by excision of the polynucleotide and/or regulated expression of the polypeptide. Excision of a polynucleotide encoding the polypeptide can be provided by use of site-specific recombination systems (e.g., Cre-Lox, FLP-FRT). Regulated expression of the polypeptide can be effectuated by methods including: (i) operable linkage of the polynucleotide encoding the polypeptide to a developmentally-regulated, de-repressable, and/or inducible promoter; and/or (ii) introduction of a polynucleotide (e.g., dsRNA or amiRNA) that can induce siRNA-mediated inhibition of the polypeptide. Suitable site-specific recombination systems as well as developmentally-regulated, de-repressable, and/or inducible promoters include those disclosed in US Patent Application Publication No. 20170121722, which is incorporated herein by reference in its entirety and specifically with respect to such disclosure. Polynucleotides that can be used to effectuate transient expression of the WUS/WOX polypeptide include: (a) double-stranded RNA; (b) single-stranded RNA; (c) chemically modified RNA; (d) double-stranded DNA; (e) single-stranded DNA; (f) chemically modified DNA; or (g) a combination of (a)-(f). Certain embodiments of the polynucleotide further include additional nucleotide sequences that provide useful functionality; non-limiting examples of such additional nucleotide sequences include an aptamer or riboswitch sequence, nucleotide sequence that provides secondary structure such as stem-loops or that provides a sequence-specific site for an enzyme (e.g., a sequence-specific recombinase or endonuclease site), T-DNA (e.g., DNA sequence encoding a WUS/WOX polypeptide is enclosed between left and right T-DNA borders from Agrobacterium spp. or from other bacteria that infect or induce tumors in plants), a DNA nuclear-targeting sequence, a regulatory sequence such as a promoter sequence, and a transcript-stabilizing or -destabilizing sequence. Certain embodiments of the polynucleotide include those wherein the polynucleotide is complexed with, or covalently or non-covalently bound to, a non-nucleic acid element, e.g., a carrier molecule, an antibody, an antigen, a viral movement protein, a cell-penetrating or pore-forming peptide, a polymer, a detectable label, a quantum dot, or a particulate or nanoparticulate.

Transient expression of the WUS/WOX polypeptide can be for a period of time and/or in an amount sufficient to result in improved regenerative potential in comparison to a control plant cell. In certain embodiments, the transient increase in the expression of the WUS/WOX polypeptide is for a period of about 1, 2, 4, 8, 12, 16, 20, 24, 30, or 36 hours to about 72, 96, 120, 144, 168, 192, 276, or 336 hours. In certain embodiments, the transient increase in the expression of WUS/WOX polypeptide is for a period of about 2, 4, 8, 12, or 16 hours to about 18, 20, 24, 30 or 36 hours. In certain embodiments, the transient increase in the expression of WUS/WOX polypeptide is for a period of about 18, 20, 24, 30 or 36 hours to about 60, 80, 100, 120, 168, or 192 hours. Such transient increases in expression of WUS/WOX polypeptide can be measured by methods whereby accumulated gene products including mRNAs and/or proteins are measured. Useful methods of measuring mRNAs include quantitative reverse transcriptase Polymerase Chain Reaction (qRT-PCR)-based and/or any hybridization-based assay. Useful methods for quantitating proteins include immunoassays (e.g., ELISAs, RIAs) and/or mass spectrometry-based methods.

Genome editing systems of use in the methods provided herein include molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA or donor or other DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas12L, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) donor DNA template polynucleotides suitable for insertion at a break in genomic DNA by homology-directed repair (HDR) or microhomology-mediated end joining (MMEJ); and (e) other DNA templates (e.g., dsDNA, ssDNA, or combinations thereof) suitable for insertion at a break in genomic DNA (e.g., by non-homologous end joining (NHEJ).

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to US Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). In certain embodiments, an RNA-guided endonuclease that leaves a blunt end following cleavage of the target site is used. Blunt-end cutting RNA-guided endonucleases include Cas9, Cas12c, Cas12i, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e. All of the patent publications referenced in this paragraph are incorporated herein by reference in their entirety.

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, plants or plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least 16 or 17 nucleotides of gRNA sequence are required by Cas9 for DNA cleavage to occur; for Cpf1 at least 16 nucleotides of gRNA sequence are needed to achieve detectable DNA cleavage and at least 18 nucleotides of gRNA sequence were reported necessary for efficient DNA cleavage in vitro; see Zetsche et al. (2015) Cell, 163:759-771. In practice, guide RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., a gRNA with a length of 20 nucleotides and 1-4 mismatches to the target sequence) but can increase the potential for off-target effects. The design of effective guide RNAs for use in plant genome editing is disclosed in US Patent Application Publication 2015/0082478 A1, the entire specification of which is incorporated herein by reference. Efficient gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991. The design of effective gRNAs for use in plant genome editing is disclosed in US Patent Application Publication 2015/0082478 A1, the entire specification of which is incorporated herein by reference.

Other nucleases capable of effecting site-specific modification of a target nucleotide sequence in the systems, methods, and compositions provided herein include zinc-finger nucleases (ZFNs), transcription activator-like effector nucleases (TAL-effector nucleases or TALENs), Argonaute proteins, and a meganuclease or engineered meganuclease. Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see, e.g., Umov et al. (2010) Nature Rev. Genet., 11:636-646. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally-occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) are well known and described in the literature. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fokl. This endonuclease must dimerize to cleave DNA. Thus, cleavage by Fokl as part of a ZFN requires two adjacent and independent binding events, which must occur in both the correct orientation and with appropriate spacing to permit dimer formation. The requirement for two DNA binding events enables more specific targeting of long and potentially unique recognition sites. Fokl variants with enhanced activities have been described; see, e.g., Guo et al. (2010) J. Mol. Biol., 400:96-107.

Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fokl, can be conveniently used. For a description of the use of TALENs in plants, see Mahfouz et al. (2011) Proc. Natl. Acad. Sci. USA, 108:2623-2628 and Mahfouz (2011) GM Crops, 2:99-103.

Argonautes are proteins that can function as sequence-specific endonucleases by binding a polynucleotide (e.g., a single-stranded DNA or single-stranded RNA) that includes sequence complementary to a target nucleotide sequence) that guides the Argonaut to the target nucleotide sequence and effects site-specific alteration of the target nucleotide sequence; see, e.g., US Patent Application Publication 2015/0089681, incorporated herein by reference in its entirety.

In related embodiments, zinc finger nucleases, TALENs, and Argonautes are used in conjunction with other functional domains. For example, the nuclease activity of these nucleic acid targeting systems can be altered so that the enzyme binds to but does not cleave the DNA. Examples of functional domains include transposase domains, integrase domains, recombinase domains, resolvase domains, invertase domains, protease domains, DNA methyltransferase domains, DNA hydroxylmethylase domains, DNA demethylase domains, histone acetylase domains, histone deacetylase domains, nuclease domains, repressor domains, activator domains, nuclear-localization signal domains, transcription-regulatory protein (or transcription complex recruiting) domains, cellular uptake activity associated domains, nucleic acid binding domains, antibody presentation domains, histone modifying enzymes, recruiter of histone modifying enzymes; inhibitor of histone modifying enzymes, histone methyltransferases, histone demethylases, histone kinases, histone phosphatases, histone ribosylases, histone deribosylases, histone ubiquitinases, histone deubiquitinases, histone biotinases and histone tail proteases. Non-limiting examples of functional domains include a transcriptional activation domain, a transcription repression domain, and an SHH1, SUVH2, or SUVH9 polypeptide capable of reducing expression of a target nucleotide sequence via epigenetic modification; see, e.g., US Patent Application Publication 2016/0017348, incorporated herein by reference in its entirety. Genomic DNA may also be modified via base editing using a fusion between a catalytically inactive Cas9 (dCas9) fused to a cytidine deaminase which converts cytosine (C) to uridine (U), thereby effecting a C to T substitution; see Komor et al. (2016) Nature, 533:420-424. In other embodiments, adenine base editors (ABEs) can be used to convert A/T base pairs to G/C base pairs in genomic DNA (Gaudelli et al., 2017).

Other genome altering reagents used in plant cells and methods provided herein include transgenes or vectors comprising the same. Such transgenes can confer useful traits that include herbicide tolerance, pest tolerance (e.g., tolerance to insects, nematodes, or plant pathogenic fungi and bacteria), improved yield, increased and/or qualitatively improved oil, starch, and protein content, improved abiotic stress tolerance (e.g., improved or enhanced water use efficiency or drought tolerance, osmotic stress tolerance, high salinity stress tolerance, heat stress tolerance, enhanced cold tolerance, including cold germination tolerance), and the like. Such transgenes include both transgenes that confer the trait by expression of an exogenous protein as well as transgenes that confer the trait by inhibiting expression of endogenous plant genes (e.g., by inducing an siRNA response which inhibits expression of the endogenous plant genes). Transgenes that can provide such traits are disclosed in US Patent Application Publication Nos. 20170121722 and 20170275636, which are each incorporated herein by reference in their entireties and specifically with respect to such disclosures.

In some embodiments, one or more polynucleotides or vectors driving expression of one or more polynucleotides encoding any of the WUS/WOX polypeptides and/or genome editing systems are introduced into a plant cell. In certain embodiments, a polynucleotide vector comprises a regulatory element such as a promoter operably linked to one or more polynucleotides encoding the polypeptide or genome editing system. In such embodiments, expression of these polynucleotides can be controlled by selection of the appropriate promoter, particularly promoters functional in a plant cell; useful promoters include constitutive, conditional, inducible, and temporally or spatially specific promoters (e.g., a tissue specific promoter, a developmentally regulated promoter, or a cell cycle regulated promoter). Developmentally regulated promoters that can be used include Phospholipid Transfer Protein (PLTP), fructose-1,6-bisphosphatase protein, NAD(P)-binding Rossmann-Fold protein, adipocyte plasma membrane-associated protein-like protein, Rieske [2Fe-2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase protein that are disclosed in US Patent Application Publication No. 20170121722, which is incorporated herein by reference in its entirety and specifically with respect to such disclosure. In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see, e.g., Ferré-D'Amare and Scott (2014) Cold Spring Harbor Perspectives Biol., 2:a003574). In certain embodiments, the promoter is an RNA polymerase III promoter operably linked to a nucleotide sequence encoding one or more guide RNAs. In certain embodiments, the promoter operably linked to one or more polynucleotides is a constitutive promoter that drives gene expression in plant cells. In certain embodiments, the promoter drives gene expression in the nucleus or in an organelle such as a chloroplast or mitochondrion. Examples of constitutive promoters include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PPDK) promoter, which is active in photosynthetic tissues. Other contemplated promoters include cell-specific or tissue-specific or developmentally regulated promoters, for example, a promoter that limits the expression of the nucleic acid targeting system to germline or reproductive cells (e.g., promoters of genes encoding DNA ligases, recombinases, replicases, or other genes specifically expressed in germline or reproductive cells). In certain embodiments, the genome alteration is limited only to those cells from which DNA is inherited in subsequent generations, which is advantageous where it is desirable that expression of the genome-editing system be limited in order to avoid genotoxicity or other unwanted effects. All of the patent publications referenced in this paragraph are incorporated herein by reference in their entirety.

Expression vectors or polynucleotides provided herein may contain a DNA segment near the 3′ end of an expression cassette that acts as a signal to terminate transcription and directs polyadenylation of the resultant mRNA, and may also support promoter activity. Such a 3′ element is commonly referred to as a “3′-untranslated region” or “3′-UTR” or a “polyadenylation signal.” In some cases, plant gene-based 3′ elements (or terminators) consist of both the 3′-UTR and downstream non-transcribed sequence (Nuccio et al., 2015). Useful 3′ elements include: Agrobacterium tumefaciens nos 3′, tml 3′, tmr 3′, tins 3′, ocs 3′, and tr7 3′ elements disclosed in U.S. Pat. No. 6,090,627, incorporated herein by reference, and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1, incorporated herein by reference.

In certain embodiments, a vector or polynucleotide comprising an expression cassette includes additional components, e.g., a polynucleotide encoding a drug resistance or herbicide gene or a polynucleotide encoding a detectable marker such as green fluorescent protein (GFP) or beta-glucuronidase (gus) to allow convenient screening or selection of cells expressing the vector or polynucleotide. Selectable markers include genes that confer resistance to herbicidal compounds, such as glyphosate, sulfonylureas, glufosinate ammonium, bromoxynil, imidazolinones, and 2,4-dichlorophenoxyacetate (2,4-D). Since the expression of WUS/WOX genes can accelerate somatic embryogenesis and embryo maturation, selectable marker genes, selective agents, and conditions can be adjusted to minimize formation of un-edited or untransformed regenerable plant structures (e.g., “escapes”). Such selectable marker genes and selective agents include the maize HRA gene (Lee et al., 1988, EMBO J 7:1241-1248) which confers resistance to sulfonylureas and imidazolinones, the CP4 gene that confers resistance to glyphosate (US Reissue Patent RE039247, specifically incorporated herein by reference in its entirety and with respect to such genes and related selection methods), the GAT gene which confers resistance to glyphosate (Castle et al., 2004, Science 304:1151-1154), genes that confer resistance to spectinomycin such as the aadA gene (Svab et al., 1990, Plant Mol Biol. 14:197-205) and the bar gene that confers resistance to glufosinate ammonium (White et al., 1990, Nucl. Acids Res. 25:1062), and PAT (or moPAT for corn, see Rasco-Gaunt et al., 2003, Plant Cell Rep. 21:569-76; also see Sivamani et al., 2019) and the PMI gene that permits growth on mannose-containing medium (Negrotto et al., 2000, Plant Cell Rep. 22:684-690).

### EMBODIMENTS

The following numbered embodiments also form part of the present disclosure:

1. A method of producing a regenerable plant structure, the method comprising: introducing a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37 in a monocot plant cell; and culturing the monocot plant cell to produce the regenerable plant structure, optionally wherein the monocot plant cell is a maize plant cell.

2. The method of embodiment 1, wherein the polynucleotide encoding the polypeptide has at least 90%, at least 95%, at least 96%, at least 97%, at least 98%, at least 99%, or at least 99.5% sequence identity across the entire length of SEQ ID NO: 7, 8, 9, 36, or 38.

3. The method of embodiment 1 or embodiment 2, wherein the polynucleotide is operably linked to a heterologous promoter functional in a plant cell.

4. The method of any one of embodiments 1-3, wherein the polynucleotide is stably incorporated into the genome of the monocot plant cell, optionally wherein the monocot plant cell is a maize plant cell.

5. The method of any one of embodiments 1-3, wherein the polynucleotide is transiently expressed in the monocot plant cell, optionally wherein the monocot plant cell is a maize plant cell.

6. The method of any one of embodiments 1-5, wherein the polynucleotide encodes a polypeptide comprising the amino acid sequence of SEQ ID NO: 1, 3, 17, 18, 19, 20, 21, 22, 23, 24, or 25, or an allelic variant thereof.

7. The method of any one of embodiments 1-5, wherein the polynucleotide encodes a polypeptide comprising the amino acid sequence of SEQ ID NO: 2, 26, 27, 29, 30, 31, 32, 33, or 34, or an allelic variant thereof.

8. The method of any one of embodiments 1-5, wherein the polynucleotide encodes a polypeptide comprising the amino acid sequence of SEQ ID NO: 1, 2, 3, 35, or 37, or an allelic variant thereof.

9. The method of any one of embodiments 1-8, further comprising introducing a polynucleotide encoding a polypeptide having at least 90%, at least 95%, at least 96%, at least 97%, at least 98%, at least 99%, or at least 99.5% sequence identity across the entire length of SEQ ID NO: 5 or 6 in the maize plant cell.

10. The method of any one of embodiments 1-9, wherein the monocot plant cell comprises a regeneration-recalcitrant germplasm, optionally wherein the monocot plant cell is a maize plant cell.

11. The method of any one of embodiments 1-10, wherein the regenerable plant structure comprises a somatic embryo, embryogenic callus, somatic meristem, organogenic callus, a shoot, or a shoot further comprising roots.

12. The method of any one of embodiments 1-11, wherein the introducing comprises bacterial-mediated transformation or biolistic-mediated transformation.

13. The method of any one of embodiments 1-12, wherein expression of the polypeptide results in an increased somatic embryo induction frequency or increased embryo productivity relative to a control monocot plant cell lacking the polynucleotide encoding the polypeptide, optionally wherein the monocot plant cell is a maize plant cell.

14. The method of embodiment 13, wherein the somatic embryo induction frequency is increased at least about 50%, at least about 60%, at least about 70%, at least about 80%, at least about 90%, or at least about 95%, optionally wherein the somatic embryo induction frequency is increased at least 2-fold, at least 5-fold, at least 10-fold, or at least 20-fold relative to a control monocot plant cell lacking the polynucleotide encoding the polypeptide.

15. The method of embodiment 13 or embodiment 14, wherein the embryo productivity is increased to an assigned embryo productivity score of at least about 1, at least about 1.5, at least about 2, or at least about 2.5 somatic embryos per embryogenic immature embryo, optionally wherein the embryo productivity is increased to an assigned embryo productivity score of about 1 to about 3 or from about 1.5 to about 2.5 somatic embryos per embryogenic immature embryo.

16. The method of any one of embodiments 1-15, further comprising introducing a genome editing system in the monocot plant cell, optionally wherein the monocot plant cell is a maize plant cell.

17. The method of embodiment 16, wherein the genome editing system comprises a CRISPR-based system, a transcription activator-like effector nuclease (TALEN) system, or a zinc finger nuclease (ZFN) system, and optionally a donor template polynucleotide.

18. The method of embodiment 17, wherein the CRISPR-based system comprises (i) an RNA-guided nuclease or a polynucleotide encoding the RNA-guided nuclease; and (ii) a guide RNA or a polynucleotide encoding the gRNA.

19. A monocot plant cell comprising a heterologous polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37, wherein expression of the polypeptide increases proliferation, somatic embryogenesis, and/or regeneration capacity of the maize plant cell, optionally wherein the monocot plant cell is a maize, wheat, or rice plant cell.

20. The monocot plant cell of embodiment 19, wherein the polynucleotide encoding the polypeptide has at least 90%, at least 95%, at least 96%, at least 97%, at least 98%, at least 99%, or at least 99.5% sequence identity across the entire length of SEQ ID NO: 7, 8, 9, 36, or 38.

21. The monocot plant cell of embodiment 19 or embodiment 20, wherein the polynucleotide is operably linked to a heterologous promoter functional in a plant cell.

22. The monocot plant cell of any one of embodiments 19-21, wherein the polynucleotide is stably incorporated into the genome of the monocot plant cell.

23. The monocot plant cell of any one of embodiments 19-22, wherein the polynucleotide is transiently expressed in the monocot plant cell.

24. The monocot plant cell of any one of embodiments 19-22, wherein the polynucleotide comprises an mRNA.

25. The monocot plant cell of any one of embodiments 19-24, wherein the polynucleotide encodes a polypeptide comprising the amino acid sequence of SEQ ID NO: 1, 3, 17, 18, 19, 20, 21, 22, 23, 24, or 25, or an allelic variant thereof.

26. The monocot plant cell of any one of embodiments 19-24, wherein the polynucleotide encodes a polypeptide comprising the amino acid sequence of SEQ ID NO: 2, 26, 27, 29, 30, 31, 32, 33, or 34, or an allelic variant thereof.

27. The monocot plant cell of any one of embodiments 19-24, wherein the polynucleotide encodes a polypeptide comprising the amino acid sequence of SEQ ID NO: 1, 2, 3, 35, or 37, or an allelic variant thereof.

28. The monocot plant cell of any one of embodiments 19-27, further comprising a polynucleotide encoding a polypeptide having at least 90%, at least 95%, at least 96%, at least 97%, at least 98%, at least 99%, or at least 99.5% sequence identity across the entire length of SEQ ID NO: 5 or 6.

29. The monocot plant cell of any one of embodiments 19-28, wherein the monocot plant cell comprises a regeneration-recalcitrant germplasm.

30. The monocot plant cell of any one of embodiments 19-20, wherein expression of the polypeptide results in an increased somatic embryo induction frequency or increased embryo productivity relative to a control monocot plant cell lacking the polynucleotide encoding the polypeptide.

31. The monocot plant cell of embodiment 30, wherein the somatic embryo induction frequency is at least about 50%, at least about 60%, at least about 70%, at least about 80%, at least about 90%, or at least about 95%, optionally wherein the somatic embryo induction frequency is increased at least 2-fold, at least 5-fold, at least 10-fold, or at least 20-fold relative to a control monocot plant cell lacking the polynucleotide encoding the polypeptide.

32. The monocot plant cell of embodiment 30 or embodiment 31, wherein the embryo productivity is increased to an assigned embryo productivity score of at least about 1, at least about 1.5, at least about 2, or at least about 2.5 somatic embryos per embryogenic immature embryo, optionally wherein the embryo productivity is increased to an assigned embryo productivity score of about 1 to about 3 or from about 1.5 to about 2.5 somatic embryos per embryogenic immature embryo.

33. The monocot plant cell of any one of embodiments 19-32, further comprising a genome editing system.

34. The monocot plant cell of embodiment 33, wherein the genome editing system comprises a CRISPR-based system, a transcription activator-like effector nuclease (TALEN) system, or a zinc finger nuclease (ZFN) system, and optionally a donor template polynucleotide.

35. The monocot plant cell of embodiment 34, wherein the CRISPR-based system comprises (i) a RNA-guided nuclease or a polynucleotide encoding the RNA-guided nuclease; and (ii) a guide RNA or a polynucleotide encoding the gRNA.

36. A monocot plant, tissue, organ, callus, or cell culture comprising the monocot plant cell of any one of embodiments 19-35.

37. A recombinant polynucleotide comprising: (a) a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37 or (b) a polynucleotide having at least 90%, at least 95%, at least 96%, at least 97%, at least 98%, at least 99%, or at least 99.5% sequence identity across the entire length of SEQ ID NO: 7, 8, 9, 36, or 38; optionally wherein the polynucleotide of (a) or (b) is operably linked to a heterologous promoter functional in a plant cell.

38. The recombinant polynucleotide of embodiment 37, wherein the polynucleotide encodes a polypeptide comprising the amino acid sequence of SEQ ID NO: 1, 3, 17, 18, 19, 20, 21, 22, 23, 24, or 25, or an allelic variant thereof.

39. The recombinant polynucleotide of embodiment 37 or embodiment 38, wherein the polynucleotide encodes a polypeptide comprising the amino acid sequence of SEQ ID NO: 2, 26, 27, 29, 30, 31, 32, 33, or 34 or an allelic variant thereof.

40. The recombinant polynucleotide of any one of embodiments 37-39, wherein the polynucleotide encodes a polypeptide comprising the amino acid sequence of SEQ ID NO: 1, 2, 3, 35, or 37, or an allelic variant thereof.

41. A vector comprising the recombinant polynucleotide of any one of embodiments 37-40.

42. A method for producing a monocot plant, the method comprising: regenerating a monocot plant from the monocot plant cell of any one of embodiments 19-35.

43. The method of embodiment 42 further comprising selecting a progeny of the monocot plant that lacks the polynucleotide.

44. The method of embodiment 42 or embodiment 43, wherein the monocot plant comprises an inserted transgene, a target gene edit, or a genome edit.

### EXAMPLES

### Example 1

This example describes the methods used in the experiments of Examples 2 to 5.

### Explants

Immature embryos of GIDA9924 or GIJ19901 were isolated 9-13 days after pollination.

### Transformation

Agrobacterium-mediated transformation was performed essentially by conventional methods (see U.S. Pat. Nos. 5,591,616, 7,939,328), using DL-phosphinothricin (PPT) as a selection agent. The transformation steps were Embryo Inoculation (1 day), Coculture (3 days), Resting (7 days), Selection 1 (7 days), Selection 2 (14 days), Selection 3 (7 days), Pre-Regeneration (5-7 days), Regeneration (14 days), and Shoot Growth (1-3 weeks), followed by transplant to soil.

### Vectors

Binary T-DNA vectors with the following relevant expression cassettes between left and right borders were used in the experiments.

TABLE 1

| ID | Constructs |
|---|---|
| pIN2528 | 35Sp::PAT::NOSt; ZmUbi1p::mScarletNLS::ZmUbi1t |
| pIN2541 | 35Sp::PAT::NOSt; ZmUbi1p::mScarletNLS::ZmUbi1t |
| pIN3239 | 35Sp::PAT::35S-NOSt; OsAct1p::mScarlet::OsAct1t; |
| pIN3250 | 35Sp::PAT::35S-NOSt; ZmUbi1p::SbWox5::ZmUbi1t; |
| | OsAct1p::mScarlet::OsAct1t |
| pIN3261 | 35Sp::PAT::35S-NOSt; ZmUbi1p::SbWox5::ZmUbi1t; |
| | OsAct1p::mScarlet::OsAct1t |
| pIN3262 | 35Sp::PAT::35S-NOSt; NOSp::SbWox5::NOS-35St; |
| | ZmUbi1::MUT_PhBBM2:: ZmUbi1t; |
| | OsAct1p::mScarlet::OsAct1t |
| pIN3263 | 35Sp::PAT::35S-NOSt; NOSp::SbWox5::NOS-35St; |
| | ZmUbi1::MUT_SiBBM2:: ZmUbi1t; |
| | OsAct1p::mScarlet::OsAct1t |
| pIN3258 | 35Sp::PAT::35S-NOSt; ZmUbi1p::MlWUS2::ZmUbi1t; |
| | OsAct1p::mScarlet::OsAct1t |
| pIN3259 | 35Sp::PAT::35S-NOSt; NOSp::MlWus2::NOS-35St; |
| | ZmUbi1p::PhBBM2::ZmUbi1t; OsAct1p::mScarlet::OsAct1t |
| pIN3260 | 35Sp::PAT::35S-NOSt; NOSp::MlWus2::NOS-35St; |
| | ZmUbi1::MUT_SiBBM2:: ZmUbi1t; |
| | OsAct1p::mScarlet::OsAct1t |
| pIN3267 | 35Sp::PAT::35S-NOSt; ZmUbi1::SiWOX4:: ZmUbi1t; |
| | OsAct1p::mScarlet::OsAct1t |
| pIN3268 | 35Sp::PAT::35S-NOSt; ZmUbi1::DoWOX2:: ZmUbi1t; |
| | OsAct1p::mScarlet::OsAct1t |
| pIN3956 | 35Sp::PAT::35S-NOSt; ZmUbi1p::PhWOX2.2:: ZmUbi1t; |
| | ZmEF1ap::mScarlet::ZmEF1at |
| pIN3955 | 35Sp::PAT::35S-NOSt; |
| | ZmUbip::MlWox2.2::ZmUbit; ZmEf1ap::mScarlet::ZmEF1at |
| pIN3759 | 35Sp::PAT::35S-NOSt; ZmEF1ap::MlWUS2::ZmEF1at; |
| pIN3760 | 35Sp::PAT::35S-NOSt; ZmEF1ap::SbWOX5::ZmEF1at; |
| pIN3761 | 35Sp::PAT::35S-NOSt |

p = promoter
t = 3′UTR and terminator
Act = actin
EF = elongation factor
mScarlet = red fluorescent marker

### Example 2

Immature zygotic embryo explants of corn genotype GIDA9924 were transformed with Agrobacterium LBA4404 carrying one of plasmids pIN3261, pIN3262, pIN3263, pIN3239, or pIN2541 as indicated.

Expression of the fluorescent marker mScarlet indicated successful transformation at the time of transfer to the resting stage.

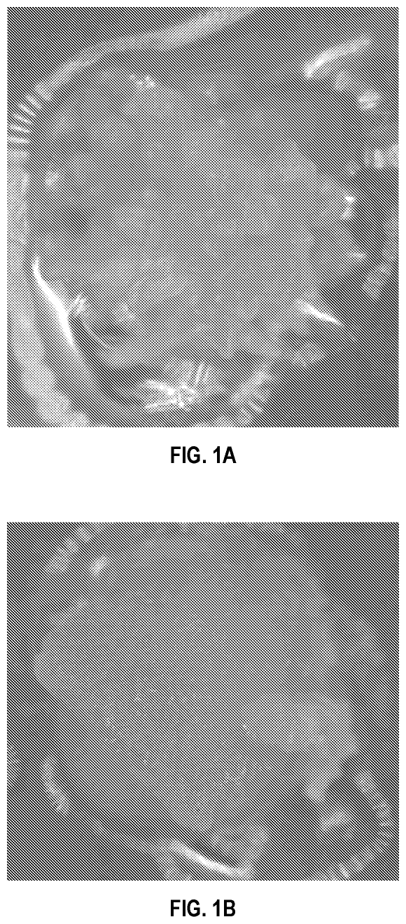

One week later, the cultured embryogenic calli were photographed and somatic embryogenesis responses were recorded. pIN3261-transformed materials had a significant amount of SEs, whereas pIN3239 or pIN3262 had fewer (FIG. 1A-B).

Somatic embryos from all construct-treatments except for pIN3261 are heterogenous with a portion that looks similar to those of GTDA9924 in media with typical appearance of early embryos.

pIN3261 showed drastic improvement of somatic embryogenesis (SE) as compared with pIN3239 empty control and other constructs. Its average somatic embryo induction frequency is high up to 94.2%, in sharp contrast with pIN3239 control which is only 16.7%. This is 5.7-fold increase. (SE induction frequency (%)=(the number of immature embryos producing somatic embryos divided by the number of immature embryos to start with)×100). Its embryo productivity is 1.9 which is also higher than empty control of 1.1. pIN3263 also showed improved SE compared with pIN3239 empty control with average somatic embryo induction frequency 35.9% which is 2.2-fold increase than empty control. Both constructs showed consistent improvement in SE across three ears.

### Example 3

Immature zygotic explants of corn genotype GIDA9924 were transformed with Agrobacterium strain LBA4404 carrying one of plasmids pIN3258, pIN3259, pIN3260, pIN3239, or pIN2541 as indicated.

Expression of the fluorescent marker mScarlet indicated successful transformation at the time of transfer to the resting stage.

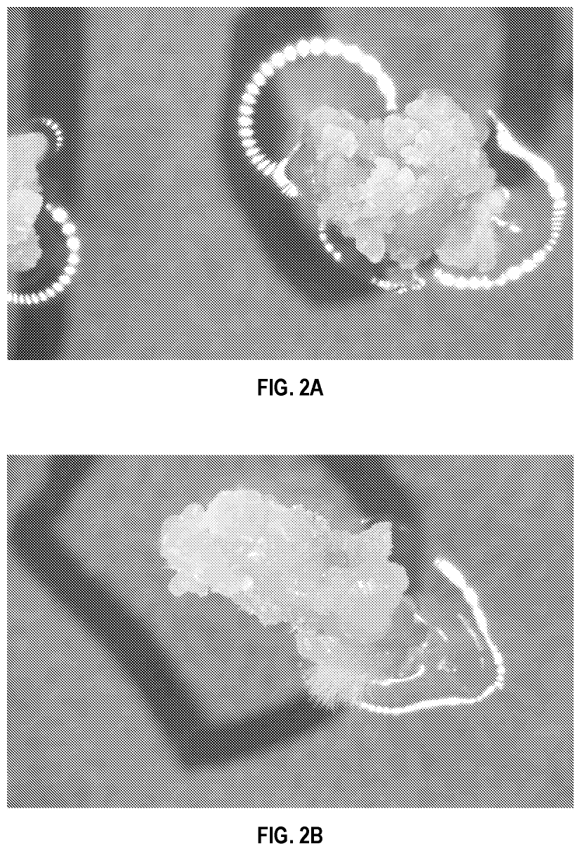

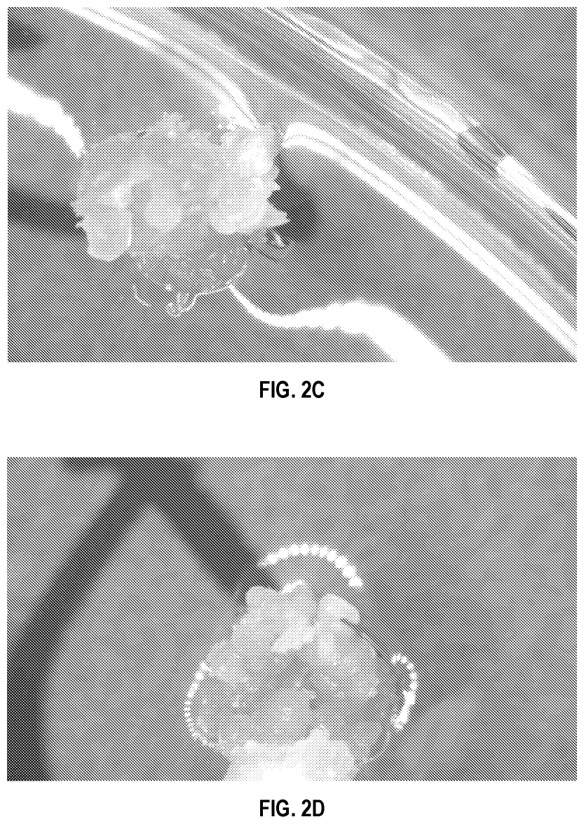

One week later, the cultured embryogenic calli were photographed and somatic embryogenesis responses were recorded. pIN3258-transformed immature embryo explants had a significant amount of SEs, whereas pIN3239 or pIN3262 had fewer (FIG. 2A-D).

Somatic embryos from all construct-treatments are heterogenous with a portion that looks similar to those of GIDA9924 in media with typical appearance of early embryos.

pIN3258 showed drastic improvement of somatic embryogenesis (SE) as compared with pIN3239 empty control and other constructs. Its average somatic embryo induction frequency over 3 ears with 120 immature embryos is high up to 83.3%, in contrast with pIN3239 control which is only 58.8%. This is 1.4-fold increase. Its embryo productivity is 1.6 which is also higher than empty control of 1.2.

### Example 4

In this experiment, the constructs pIN3258 and pIN3261 were tested on corn highly recalcitrant genotype GIJI9901 for improved SE.

Immature zygotic embryo explants of corn genotype GIJI9901 were transformed with Agrobacterium strain LBA4404 carrying one of plasmids pIN3258, pIN3261, pIN3239, or pIN2541 as indicated.

Expression of the fluorescent marker mScarlet indicated successful transformation at the time of transfer to the resting stage.

One week later, the cultured embryogenic calli were photographed and somatic embryogenesis responses were recorded. pIN3258-transformed immature embryo explants had a drastic amount of SEs, whereas pIN3239 or pIN3262 had much fewer.

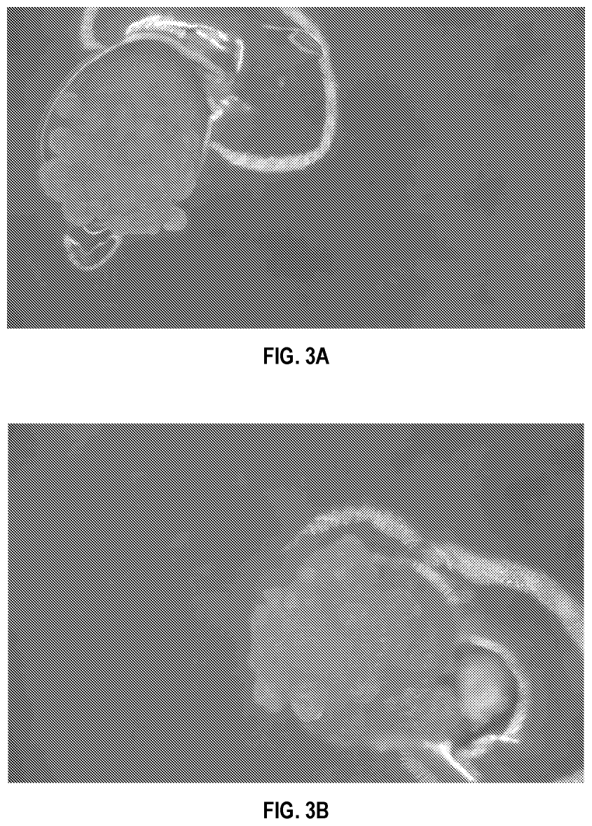

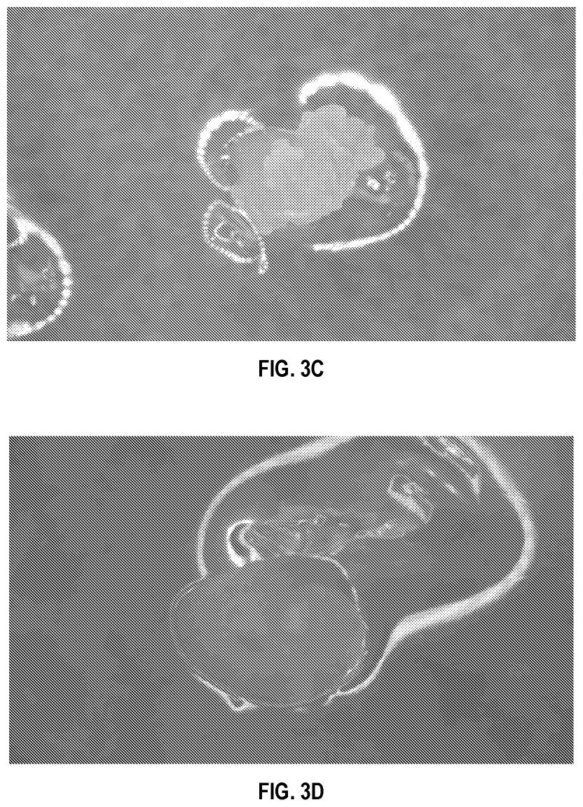

Somatic embryogenesis responses and photos (FIG. 3A-D) and were taken at the end of Rest stage (10 days after zygotic embryo isolation and infection with Agrobacterium). pIN3258 showed a drastic improvement of somatic embryogenesis (SE) as compared with pIN3239 empty control. Its average somatic embryo induction frequency over 3 ears with 120 immature embryos is high up to 91.1%, in contrast with pIN3239 control which is only 5.0%. This is 18.8-fold increase. Its embryo productivity is 1.3 which is also higher than empty control of 1.0. pIN3261 also showed a drastic improvement of somatic embryogenesis (SE) as compared with pIN3239 empty control. Its average somatic embryo induction frequency over 3 ears with 120 immature embryos is high up to 52.2%, in contrast with pIN3239 control which is only 5.0%. This is an over 10-fold increase.

### Example 5

Immature zygotic embryo explants of corn genotype GTDA9924 were transformed with Agrobacterium strain LBA4404 carrying one of plasmids pIN3267, pIN3268, pIN3239, or pIN2541 as indicated.

Expression of the fluorescent marker mScarlet indicated successful transformation at the time of transfer to the resting stage.

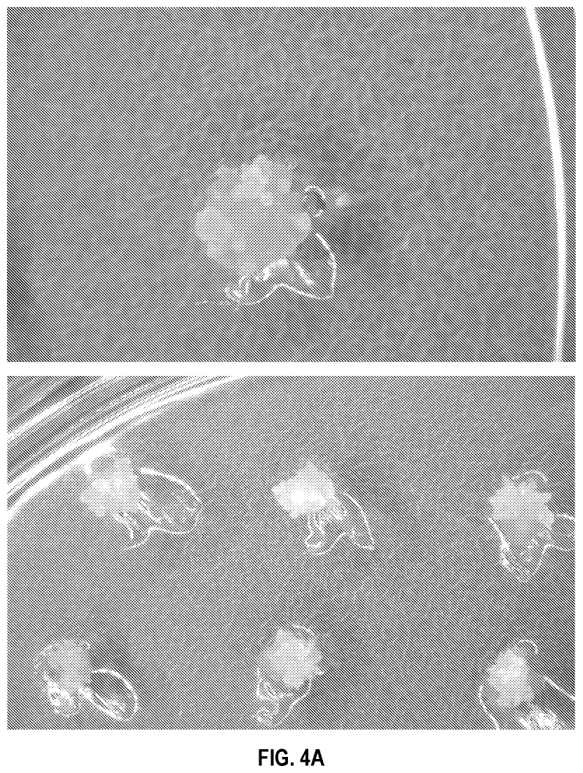

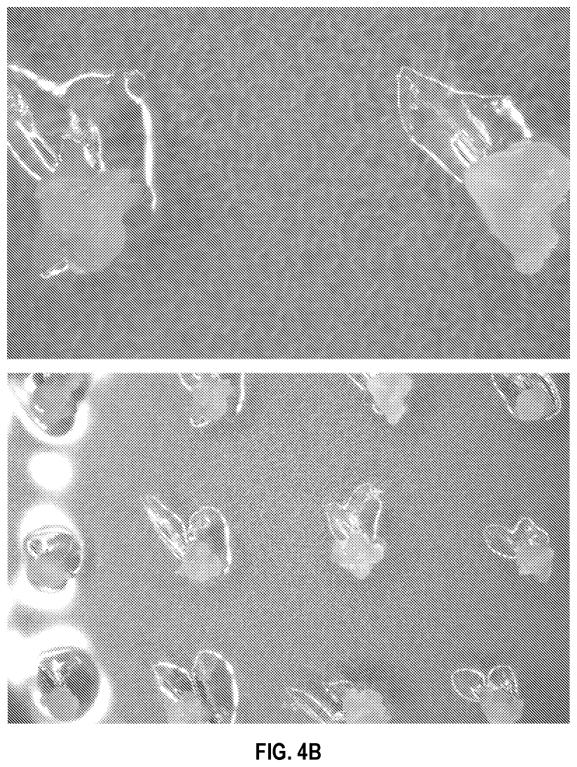

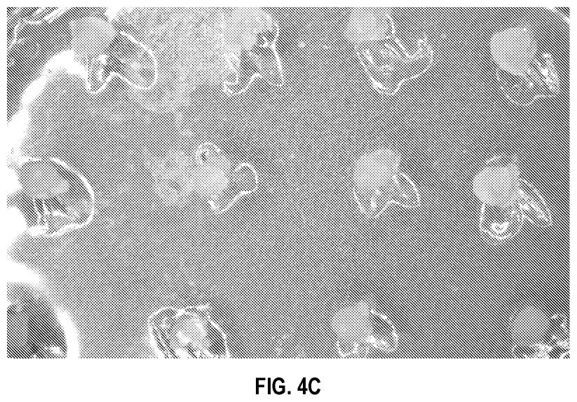

One week later, the cultured embryogenic calli were photographed and somatic embryogenesis responses were recorded. pIN3267-transformed immature embryo explants had a drastic increase of somatic embryos, whereas pIN3268 and pIN3239 had few and none, respectively (FIGS. 4A-C).

pIN3267 showed 94.2% somatic embryo induction frequency as compared with 0% in pIN3239 empty control and 7.8% in pIN3268 over 3 ears with around 120 immature embryos. The embryo productivity of pIN3267 is much higher than pIN3268 and pIN3239 which are 1.0 and 0, respectively.

The Examples demonstrate that both constructs pIN3258 and pIN3261 showed consistent improvement in SE across three ears and two different corn genotypes. In addition, construct pIN3267 showed a consistent improvement in SE across three ears in genotype GIDA9924. The somatic embryo quality in pIN3261 is better than that of pIN3258, suggesting SbWOX5 may induce a better SE quality than MlWUS2 in this corn line.

### Example 6

Immature zygotic embryo explants of corn genotype GIDA9924 were transformed with Agrobacterium strain LBA4404 carrying one of plasmids pIN3956, pIN3768, or pIN2541 as indicated.

Expression of the fluorescent marker mScarlet indicated successful transformation at the time of transfer to the resting stage.

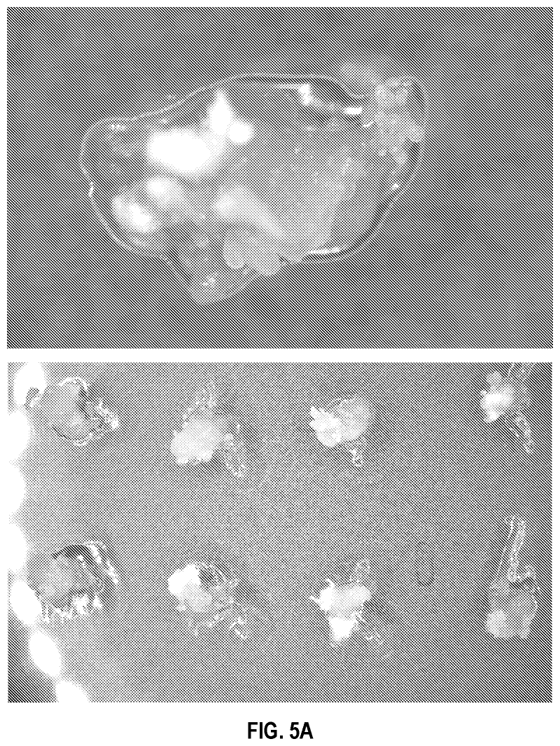

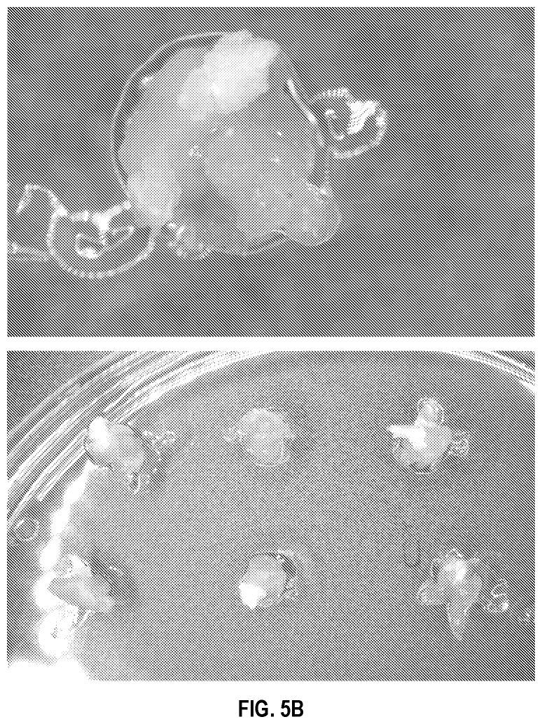

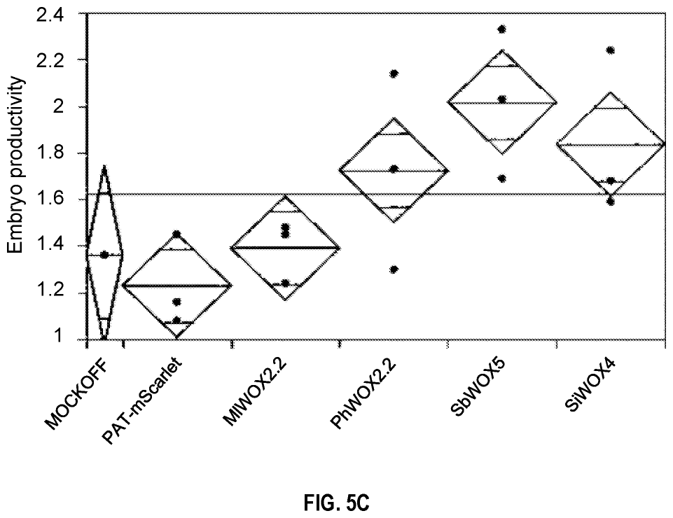

One week later, the cultured embryogenic calli were photographed and somatic embryogenesis responses were recorded. pIN3956-transformed immature embryo explants had a significant increase of somatic embryos, whereas empty control pIN3768 had few and none, respectively (FIGS. 5A-B).

pIN3956 showed 94.4% somatic embryo induction frequency as compared with 73% in pIN3768 empty control over 3 ears with around 90 immature embryos. The embryo productivity of pIN3956 is much higher than pIN3768 which are 1.8 and 1.2, respectively.

The Examples demonstrate that construct pIN3956 showed consistent improvement in SE across three ears. The somatic embryo quality in pIN3956 is better than that of pIN3768, suggesting the SE competence of PhWOX2.2.

The improved somatic embryogenesis by PhWOX2.2 was very similar to that by SiWOX4 (FIG. 5C, Table 2). Table 2 shows the percentage of immature embryos producing somatic embryos, i.e., somatic embryo induction frequency.

TABLE 2

| Morphoregulator | Somatic Embryo Induction Frequency |
|---|---|
| MOCKOFF | 83.3% |
| PAT-mScarlet | 73.0% |
| MlWOX2.2 | 87.8% |
| PhWOX2.2 | 94.4% |
| SbWOX5 | 98.9% |
| SiWOX4 | 93.3% |

MOCKOFF is without vector control. Use of Agrobacterium infection suppresses somatic embryogenesis while use of the morphoregulator can restore and even further promotes somatic embryogenesis. In transformation experiments, the MOCKOFF control is less relevant but is used for indicating the immature embryo quality. GIDA9924 used in this screen is a highly regenerable genotype and the MOCKOFF with 83.3% somatic embryo induction frequency indicates the embryo quality was very good.

### Example 7

This example illustrated that morphoregulators improved transformation and gene editing via Agrobacterium-mediated transformation. All medium recipes were listed in Table 3. Two days before infection, 10 μl glycerol stock of Agrobacterium strain carrying morphoregulator gene-containing plasmid from ultra-freezer was inoculated into a 50 ml falcon tube that contains 10 ml YEP liquid medium (10 g/L Yeast Extract, 10 g/L Peptone, 5 g/L NaCl, 50 mg/L) placed on an orbital shaker set at 225 rpm and incubated overnight at the 28° C. Next day morning, the 10 ml overnight Agrobacterium culture was poured into 40 ml YEP liquid medium in 250 ml flask and incubated on the same shaker. About 16 hours before the day 3 infection, the Agrobacterium cell suspensions were spin-down (4000 rpm, 15 min, 4° C.) and resuspended into 250 ml flask containing 50 ml of AB minimum medium (Table 3) amended with 200 μM acetosyringone and 50 mg/L spectinomycin and shaken overnight. Early morning on the day of infection, the Agrobacterium culture was spun down (4000 rpm, 15 min, 4° C.). The supernatant was removed and the Agrobacterium infection medium with acetosvringone solution was added. The Agrobacterium cells were resuspended and the optical density (660 nm) was adjusted to about 0.25 to 1.0.

TABLE 3
Medium compositions for Agrobacterium-mediated transformation and/or editing of maize (per liter)

| | AB | | Infection | | Co Culture | | Rest | | Maturation | | Regeneration | | Rooting | |
|---|---|---|---|---|---|---|---|---|---|---|---|---|---|---|
| Composition | (20X) | | ZM9011 | | ZM9022 | | ZM9023 | | ZM9175 | | ZM9177 | | ZM9193 | |
| K₂HPO₄ | 60 | g | | | | | | | | | | | | |
| NaH₂PO₄ | 20 | g | | | | | | | | | | | | |
| NH₄Cl | 20 | g | | | | | | | | | | | | |
| MgSO₄•7H₂O | 6 | g | | | | | | | | | | | | |
| KCl | 3 | g | | | | | | | | | | | | |
| CaCl₂ | 0.2 | g | | | | | | | | | | | | |
| FeSO₄•7H₂O | 0.05 | g | | | | | | | | | | | | |
| MS salts | | | 2.16 | g | 2.16 | g | 4.3 | g | 4.3 | g | 4.3 | g | 4.3 | g |
| Eriksson | | | 1 | ml | 1 | ml | 1 | ml | | | | | | |
| modified | | | | | | | | | | | | | | |
| vitamins | | | | | | | | | | | | | | |
| (1000X) | | | | | | | | | | | | | | |
| MS vitamins | | | | | | | | | 5 | ml | | | 1 | ml |
| (1000X) | | | | | | | | | | | | | | |
| Fromm | | | | | | | | | | | 1 | ml | | |
| vitamins | | | | | | | | | | | | | | |
| (1000X) | | | | | | | | | | | | | | |
| Casamino acids | | | | | | | | | | | | | | |
| Myo-inositol | | | | | 100 | mg | 100 | mg | 100 | mg | | | 1 | g |
| Sucrose | | | 68.5 | g | 20 | g | 30 | g | 60 | g | 20 | g | 20 | g |
| Glucose | | | 36 | g | 10 | g | 10 | g | | | | | | |
| L-proline | | | 0.7 | g | 0.7 | g | 1.38 | g | 0.7 | g | | | | |
| CuSO₄•5H₂O | | | | | | | | | 1.22 | g | | | | |
| MES | | | 0.5 | | 0.5 | | 0.5 | | | | | | | |
| 2,4-D | | | 1.5 | mg | 1.5 | mg | 0.75 | mg | | | | | | |
| Dicamba | | | | | | | 1.65 | mg | | | | | | |
| Acetosyringone | | | 200 | mM | 200 | mM | 200 | mM | | | | | | |
| AgNO3 | | | | | 3.4 | mg | 3.4 | mg | | | | | | |
| Carbenicillin | | | | | | | 0.5 | g | | | | | | |
| Cefotaxime | | | | | | | | | 250 | mg | 250 | mg | | |
| Meropenem | | | | | | | 10 | mg | 10 | mg | | | | |
| Indole-3-acetic | | | | | | | | | 1 | mg | | | | |
| acid (IAA) | | | | | | | | | | | | | | |
| Thidiazuron | | | | | | | | | 0.1 | mg | | | | |
| (TDZ) | | | | | | | | | | | | | | |
| Abscisic acid | | | | | | | | | 0.1 | mg | | | | |
| (ABA | | | | | | | | | | | | | | |
| trans-zeatin | | | | | | | | | 0.5 | mg | | | | |
| (tZeatin) | | | | | | | | | | | | | | |
| 6- | | | | | | | | | 0.5 | mg | | | | |
| Benzyl- | | | | | | | | | | | | | | |
| aminopurine | | | | | | | | | | | | | | |
| (6-BAP) | | | | | | | | | | | | | | |
| L-Asparagine | | | | | | | | | | | 150 | mg | | |
| monohydrate | | | | | | | | | | | | | | |
| Indole-3-butyric | | | | | | | | | | | | | 1 | mg |
| acid (IBA | | | | | | | | | | | | | | |
| Phytagel | | | | | 3 | g | 3 | g | | | 3 | g | 2.5 | g |
| Gelrite | | | | | | | | | 2.4 | g | | | | |

| | | | | | | |
|---|---|---|---|---|---|---|
| pH | 7.0 | 5.2 | 5.8 | 5.8 | 5.6 | 5.8 |

Maize ears were surface sterilized for 20 min in 50% (v/v) bleach (8.25% sodium hypochlorite) plus 2 drops of Tween-20 followed by 3 washes in sterile water. Immature embryos (IEs) were isolated from ears and placed in 2 ml of the Agrobacterium infection medium with acetosyringone solution. The optimal size of the embryos varied based on the inbred, ranging from 1.5 to 2.0 mm. The infection solution was then drawn off and 1.8 ml of Agrobacterium suspension was added to the embryos and the tube vortexed for 5 sec or Agrobacterium solution was treated with 1% BREAKTHRU surfactant for 15 min based on inbred. The microcentrifuge tube was allowed to stand for 5 min in the hood. The suspension mixture of Agrobacterium and embryos were poured onto ZM9022 Coculture medium. The Agrobacterium suspension was drawn off and the embryos were placed with scutella side up on the media. The plates were covered with lids and placed in a box with a lid and incubated in darkness at 21° C. for 3-4 days. Embryos were then transferred to Resting medium (ZM9023) for 7-14 days at 28° C. in darkness before they were transferred to Maturation medium (ZM9174) amended with various levels of phosphinothricin (PPT), based on inbred, at 28° C. in low light (around 50 μm/sec²·m²). Two weeks later, shoots were transferred to Regeneration medium (ZM9177) with or without selection, based on inbred, at 28° C. in full light (150 μm/sec²·m²). One week later, shoots were excised and transferred to Rooting medium (ZM9193) in full light. After 2-3 weeks plantlets with roots were transferred to greenhouse and analyzed using qPCR and nanopore assays.

To evaluate the impact of morphoregulators on promoting transformation, immature zygotic embryos of corn genotype GIDA8989 (recalcitrant line) were transformed with Agrobacterium strain LBA4404 carrying one of plasmids pIN3258, pIN3759, pIN3760, pIN3761, or pIN2541 as indicated.

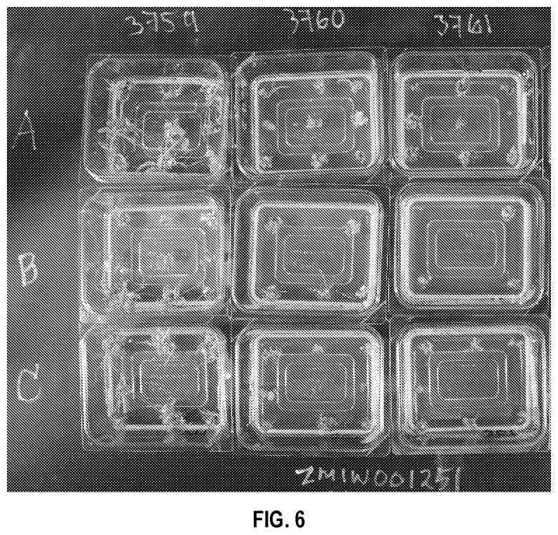

Individual regenerated events were sampled by the end of Regeneration medium and assayed by qPCR and nanopore as early detecting of the transgenic. FIG. 6 illustrated many fold increase in shoot regeneration by pIN3759 carrying MlWus2 construct as compared with pIN3760 containing SbWOX5 or pIN3761 empty control. Of the latter two constructs, pIN3760 enabled the production of more regenerants than pIN3761. Evidently, the promoting impact of SbWOX5 gene on transformation required a strong promoter such as maize pZmUBI1 because pZmEF1a promoter driving SbWOX5 showed a much lower transformation rate than the same gene driven by pZmUBI1. These improved shoot regenerations led to improved transformation efficiencies (Table 4).

TABLE 4
MlWUS2 and SbWOX5 improved transformation and/or
editing efficiencies in maize inbred lines

| | | | Number | |
|---|---|---|---|---|
| | | Number of | of qPCR | |
| | | immature | positive | Transformation |
| Genotype | Construct | embryos | events | efficiency (%) |
| GIDA8989 | pIN3759 | 150 | 19 | 12.7 |
| | pIN3760 | 150 | 0 | 0 |
| | pIN3761 | 150 | 0 | 0 |
| | pIN2541 | 20 | 0 | 0 |
| GILO8924 | pIN3759 | 270 | 6 | 2.2 |
| | pIN3760 | 150 | 0 | 0 |
| | pIN3761 | 150 | 0 | 0 |
| | pIN2541 | 20 | 0 | 0 |
| GIDA9924 | pIN3759 | 150 | 10 | 6.7 |
| | pIN3760 | 150 | 13 | 8.7 |
| | pIN3761 | 150 | 1 | 0.7 |
| | pIN2541 | 20 | 0 | 0 |
| SITU1978 | pIN3759 | 58 | 1 | 1.7 |
| | pIN4453 | 75 | 1 | 1.3 |
| | pIN2541 | 20 | 0 | 0 |
| | pIN2541 | 20 | 0 | 0 |
| GIDA8989 | pIN3760 | 4091 | 30 | 0.73 |
| | pIN2541 | 175 | 0 | 0 |

### Example 8

This example illustrated that morphoregulator SbWOX5 improved biolistics-mediated transformation efficiency. All medium compositions for particle bombardment were listed in Table 5. Immature embryos of preferred sizes, ranging from 1.5-2.0 mm, were isolated from corn ears and placed on callus induction (ZM1000) 2 days and then osmotic treatment medium (ZM2000) for three hours. Particles were prepared and the following relevant reagents were pipetted into a 2 ml low retention centrifuge tube containing 25 μl of 0.6 μm gold particles (100 μg/μl): 50 μl RNP, 4 μl plasmid (1 μg/μl concentration) carrying morphoregulator gene with or without selectable marker gene PAT, 20 μl TransIT 20/20, and 12 μl water. This solution was mixed well, spun down 30 sec at 8000 rpm, followed by removal of supernatant and adding desirable amount of water. For bombardment, 50 μl particles of the above complex was loaded onto each microcarrier, air-dried for 30-40 min until yellow dry power visible. Particle bombardment was performed using a Biolistic PDF/1000 He device at 28 inches of Mercury using a 600 PSI rupture disc.

Immature embryos of three regenerable corn genotypes GIDA9924 and GIBE0104 were transformed with biolistic PDS-1000/He device using biolistic particles and constructs pIN3250 carrying SbWOX5 and empty control constructs pIN2528 without morphogenes.

TABLE 5
Medium compositions for particle bombardment
of maize immature embryos

| | Callus | | Osmotic | | Callus | | | | | |
|---|---|---|---|---|---|---|---|---|---|---|
| | induction | | treatment | | induction | | Regeneration | | Rooting | |
| Composition | ZM1000 | | ZM2000 | | ZM3001 | | ZM4000 | | 5000 | |
| MS salts | 2.15 | g | 4.3 | g | 4.3 | g | 4.3 | g | 4.3 | g |
| MS vitamins | 0.5 | ml | 1 | ml | 1 | ml | 1 | ml | 1 | ml |
| (1000X) | | | | | | | | | | |
| Casein | | | 0.1 | g | 0.1 | g | | | | |
| hydrolysate | | | | | | | | | | |
| Myo-inositol | | | 0.1 | g | 0.1 | g | 0.1 | g | 0.1 | g |
| Sucrose | 20 | g | 30 | g | 30 | g | 60 | g | 20 | g |
| L-proline | | | 0.5 | g | 0.5 | g | | | | |
| MES | | | 0.5 | | 0.5 | | 0.5 | | | |
| Sorbitol | | | 37 | g | | | | | | |
| Mannitol | | | 37 | g | | | | | | |
| 2,4-D | | | | | 0.1 | g | 1.65 | mg | | |
| AgNO3 | | | 88 | mM | 88 | mM | 88 | mM | | |
| Gelrite | 2.5 | g | 2.5 | g | 2.5 | g | 2.5 | g | 2.5 | g |

| | | | | | |
|---|---|---|---|---|---|
| pH | 5.8 | 5.8 | 5.8 | 5.8 | 5.6 |

After particle bombardment, immature embryos were transferred to callus induction medium (ZM2000) for 16 hours, followed by callus induction media (ZM3001) for 28 days with various concentrations of PPT in either darkness or low light (based on inbred), regeneration medium (ZM4000) for 14-21 days in full light, and rooting medium (ZM5000) for 14-21 days in the full light. qPCR and nanopore were conducted on individual plantlet events by the end of regeneration as early detection of the transgenic and/or edited events. Table 6 illustrates many fold increases in transformation efficiencies by pIN3250 carrying SbWOX5 as compared with pIN2528 empty control without morphoregulator gene.

TABLE 6
Morphoregulator gene improved RNP-mediated
transformation via particle bombardment

| | | Immature | | qPCR | Regeneration | |
|---|---|---|---|---|---|---|
| | | embryos | Plants | positive | Efficiency | Transformation |
| Genotype | Construct | used | recovered | events | (%) | efficiency (%) |
| GGIBE0104 | pIN2528 | 105 | 5 | 5 | 4.8 | 4.8 |
| | pIN3250 | 140 | 14 | 11 | 10 | 10 |
| GIDA9924 | pIN2528 | 315 | 1 | 0 | 0.3 | 0.3 |
| | pIN3250 | 315 | 8 | 3 | 2.5 | 2.5 |

The breadth and scope of the present disclosure should not be limited by any of the above-described examples.

What is claimed is:

1. A method of producing a regenerable plant structure, the method comprising:
introducing a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37 in a maize plant cell; and
culturing the maize plant cell to produce the regenerable plant structure.

2. The method of claim 1, wherein the polynucleotide encoding the polypeptide has at least 90%, at least 95%, at least 96%, at least 97%, at least 98%, at least 99%, or at least 99.5% sequence identity across the entire length of SEQ ID NO: 7, 8, 9, 36, or 38.

3. The method of claim 1, wherein the polynucleotide is operably linked to a heterologous promoter functional in a plant cell.

4. The method of claim 1, wherein the polynucleotide is stably incorporated into the genome of the maize plant cell.

5. The method of claim 1, wherein the polynucleotide is transiently expressed in the maize plant cell.

6. The method of claim 1, wherein the polynucleotide encodes a polypeptide comprising the amino acid sequence of SEQ ID NO: 1 or an allelic variant thereof.

7. The method of claim 1, wherein the polynucleotide encodes a polypeptide comprising the amino acid sequence of SEQ ID NO: 2 or an allelic variant thereof.

8. The method of claim 1, wherein the polynucleotide encodes a polypeptide comprising the amino acid sequence of SEQ ID NO: 3 or an allelic variant thereof.

9. The method of claim 1, wherein the polynucleotide encodes a polypeptide comprising the amino acid sequence of SEQ ID NO: 35 or an allelic variant thereof.

10. The method of claim 1, further comprising introducing a polynucleotide encoding a polypeptide having at least 90%, at least 95%, at least 96%, at least 97%, at least 98%, at least 99%, or at least 99.5% sequence identity across the entire length of SEQ ID NO: 5 or 6 in the maize plant cell.

11. The method of claim 1, wherein the maize plant cell comprises a regeneration-recalcitrant germplasm.

12. The method of claim 1, wherein the regenerable plant structure comprises a somatic embryo, embryogenic callus, somatic meristem, organogenic callus, a shoot, or a shoot further comprising roots.

13. The method of claim 1, wherein the introducing comprises bacterial-mediated transformation or biolistic-mediated transformation.

14. The method of claim 1, wherein expression of the polypeptide results in an increased somatic embryo induction frequency or increased embryo productivity relative to a control maize plant cell lacking the polynucleotide encoding the polypeptide.

15. The method of claim 14, wherein the somatic embryo induction frequency is increased at least about 50%, at least about 60%, at least about 70%, at least about 80%, at least about 90%, or at least about 95%, optionally wherein the somatic embryo induction frequency is increased at least 2-fold, at least 5-fold, at least 10-fold, or at least 20-fold relative to a control maize plant cell lacking the polynucleotide encoding the polypeptide.

16. The method of claim 14, wherein the embryo productivity is increased to an assigned embryo productivity score of at least about 1, at least about 1.5, at least about 2, or at least about 2.5 somatic embryos per embryogenic immature embryo, optionally wherein the embryo productivity is increased to an assigned embryo productivity score of about 1 to about 3 or from about 1.5 to about 2.5 somatic embryos per embryogenic immature embryo.

17. The method of any one of claims 1 to 16, further comprising introducing a genome editing system in the maize plant cell.

18. The method of claim 17, wherein the genome editing system comprises a CRISPR-based system, a transcription activator-like effector nuclease (TALEN) system, or a zinc finger nuclease (ZFN) system, and optionally a donor template polynucleotide.

19. The method of claim 18, wherein the CRISPR-based system comprises (i) an RNA-guided nuclease or a polynucleotide encoding the RNA-guided nuclease; and (ii) a guide RNA or a polynucleotide encoding the gRNA.

20. A maize plant cell comprising a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37, wherein expression of the polypeptide increases proliferation, somatic embryogenesis, and/or regeneration capacity of the maize plant cell.

21. The maize plant cell of claim 20, wherein the polynucleotide encoding the polypeptide has at least 90%, at least 95%, at least 96%, at least 97%, at least 98%, at least 99%, or at least 99.5% sequence identity across the entire length of SEQ ID NO: 7, 8, 9, 36, or 38.

22. The maize plant cell of claim 20, wherein the polynucleotide is operably linked to a heterologous promoter functional in a plant cell.

23. The maize plant cell of claim 20, wherein the polynucleotide is stably incorporated into the genome of the maize plant cell.

24. The maize plant cell of claim 20, wherein the polynucleotide is transiently expressed in the maize plant cell.

25. The maize plant cell of claim 20, wherein the polynucleotide comprises an mRNA.

26. The maize plant cell of claim 20, wherein the polynucleotide encodes a polypeptide comprising the amino acid sequence of SEQ ID NO: 1 or an allelic variant thereof.

27. The maize plant cell of claim 20, wherein the polynucleotide encodes a polypeptide comprising the amino acid sequence of SEQ ID NO: 2 or an allelic variant thereof.

28. The maize plant cell of claim 20, wherein the polynucleotide encodes a polypeptide comprising the amino acid sequence of SEQ ID NO: 3 or an allelic variant thereof.

29. The maize plant cell of claim 20, wherein the polynucleotide encodes a polypeptide comprising the amino acid sequence of SEQ ID NO: 35 or an allelic variant thereof.

30. The maize plant cell of claim 20, further comprising a polynucleotide encoding a polypeptide having at least 90%, at least 95%, at least 96%, at least 97%, at least 98%, at least 99%, or at least 99.5% sequence identity across the entire length of SEQ ID NO: 5 or 6.

31. The maize plant cell of claim 20, wherein the maize plant cell comprises a regeneration-recalcitrant germplasm.

32. The maize plant cell of claim 20, wherein expression of the polypeptide results in an increased somatic embryo induction frequency or embryo productivity relative to a control maize plant cell lacking the polynucleotide encoding the polypeptide.

33. The maize plant cell of claim 32, wherein the somatic embryo induction frequency is increased at least about 50%, at least about 60%, at least about 70%, at least about 80%, at least about 90%, or at least about 95%, optionally wherein the somatic embryo induction frequency is increased at least 2-fold, at least 5-fold, at least 10-fold, or at least 20-fold relative to a control maize plant cell lacking the polynucleotide encoding the polypeptide.

34. The maize plant cell of claim 32, wherein the embryo productivity is increased to an assigned embryo productivity score of at least about 1, at least about 1.5, at least about 2, or at least about 2.5 somatic embryos per embryogenic immature embryo, optionally wherein the embryo productivity is increased to an assigned embryo productivity score of about 1 to about 3 or from about 1.5 to about 2.5 somatic embryos per embryogenic immature embryo.

35. The maize plant cell of any one of claims 20 to 34, further comprising a genome editing system.

36. The maize plant cell of claim 35, wherein the genome editing system comprises a CRISPR-based system, a transcription activator-like effector nuclease (TALEN) system, or a zinc finger nuclease (ZFN) system, and optionally a donor template polynucleotide.

37. The maize plant cell of claim 36, wherein the CRISPR-based system comprises (i) a RNA-guided nuclease or a polynucleotide encoding the RNA-guided nuclease; and (ii) a guide RNA or a polynucleotide encoding the gRNA.

38. A maize plant, tissue, organ, callus, or cell culture comprising the maize plant cell of any one of claims 20 to 34.

39. A recombinant polynucleotide comprising:
(a) a polynucleotide encoding a polypeptide having at least 90%, at least 95%, at least 96%, at least 97%, at least 98%, at least 99%, or at least 99.5% sequence identity across the entire length of SEQ ID NO: 1, 2, 3, 17, 18, 19, 20, 21, 22, 23, 24, 25, 26, 27, 29, 30, 31, 32, 33, 34, 35, or 37, or
(b) a polynucleotide having at least 90%, at least 95%, at least 96%, at least 97%, at least 98%, at least 99%, or at least 99.5% sequence identity across the entire length of SEQ ID NO: 7, 8, 9, 36, or 38;
wherein the polynucleotide of (a) or (b) is operably linked to a heterologous promoter functional in a plant cell.

40. The recombinant polynucleotide of claim 39, wherein the polynucleotide encodes a polypeptide comprising the amino acid sequence of SEQ ID NO: 1, 3, 17, 18, 19, 20, 21, 22, 23, 24, or 25, or an allelic variant thereof.

41. The recombinant polynucleotide of claim 39, wherein the polynucleotide encodes a polypeptide comprising the amino acid sequence of SEQ ID NO: 2, 26, 27, 29, 30, 31, 32, 33, or 34, or an allelic variant thereof.

42. The recombinant polynucleotide of claim 39, wherein the polynucleotide encodes a polypeptide comprising the amino acid sequence of SEQ ID NO: 1, 2, 3, 35, or 37, or an allelic variant thereof.

43. A vector comprising the recombinant polynucleotide of any one of claims 39 to 42.

44. A method for producing a maize plant, the method comprising:
regenerating a maize plant from the maize plant cell of any one of claims 20 to 34.

45. The method of claim 44 further comprising selecting a progeny of the maize plant that lacks the polynucleotide.

46. The method of claim 44, wherein the maize plant comprises an inserted transgene, a target gene edit, or a genome edit.
